(19) United States
(12) Patent Application Publication
(10) Pub. No.: US 2024/0139471 A1
(43) Pub. Date: May 2, 2024

(54) CATHETER

(71) Applicant: MicroVention, Inc., Aliso Viejo, CA (US)

(72) Inventors: Josiah Kessler, Mission Viejo, CA (US); Nga Ting Wong Khan, North Tustin, CA (US); Diana Nohemi Valencia, Hawaiian Gardens, CA (US)

(73) Assignee: MicroVention, Inc., Aliso Viejo, CA (US)

(21) Appl. No.: 18/496,819

(22) Filed: Oct. 27, 2023

Related U.S. Application Data
(60) Provisional application No. 63/381,529, filed on Oct. 28, 2022.

(51) Int. Cl. A61M 25/00

(52) U.S. Cl. CPC A61M 25/0053; A61M 25/0054

(57) ABSTRACT

A catheter that decreases in stiffness (i.e., increases in flexibility) toward a distal end of the catheter and then increases in stiffness (i.e., decreases in flexibility) further toward the distal end. Thus, a distal portion of the catheter, including its distal tip, may have a greater stiffness than a penultimate distal portion of the catheter immediately adjacent proximally to the distal portion. The penultimate distal portion may have a substantially uniform stiffness before transitioning to a penultimate proximal portion in which the stiffness ramps up. The penultimate proximal portion may transition to a proximal portion having a substantially uniform stiffness that is greater than the stiffnesses of the remaining portions. The manner by which the stiffness is varied along the length of the catheter may vary, including but not limited to various properties of an outer jacket, coiled wire, braided tubular mesh layer, inner coil, and/or radiopaque marker, among others.

### RELATED APPLICATIONS

This application claims priority to U.S. Provisional Application Ser. No. 63/381,529 filed Oct. 28, 2023 entitled Catheter, which is hereby incorporated herein by reference in its entirety.

### BACKGROUND OF THE INVENTION

Catheters are elongated medical devices used to access and/or help treat interior locations within a patient, such as vessels or other cavities. In some cases, catheters are advanced through blood vessels of a patient. Often such catheters may need to be relatively long to reach a desired target location within the patient. Depending on the purpose, such a catheter may be relatively small in diameter or may need to be relatively large in diameter.

In any of these cases, it is typically desirable that the catheter be both flexible enough to navigate tortuous vessel passages without damaging the vessels and have good “pushability” which allows forward movement and torquing to occur without kinking or otherwise damaging the catheter. In that respect, finding the right balance of rigidity and flexibility may sometimes vary based on the length and diameter of the catheter needed for a specific use.

### SUMMARY OF THE INVENTION

The present specification is generally directed to structural features of a catheter. Specifically, structural features that provide desirable flexibility and pushability with reduced kinking.

One aspect of the present specification is directed to a catheter that decreases in stiffness (i.e., increases in flexibility) toward a distal end of the catheter and then increases in stiffness (i.e., decreases in flexibility) further toward the distal end. For example, a distal region of the catheter may decrease in stiffness distally but increase in stiffness near or closest to the distal end of the catheter.

In one example, a catheter may include a distal region comprising a first region (e.g., length) of the catheter with a single flexibility of a first value and a second region (e.g., length) distal to the first region that has a single flexibility of a second value, lower than the first (i.e., stiffer than the first value).

In another example, a catheter may include a first region (e.g., length of catheter) that progressively increases in flexibility (i.e., decreases in stiffness) distally. In that respect, a proximal end of the first region has a lower flexibility than a distal end of the first region. A second region (e.g., length of catheter) may be located distal of the first region and is either a uniform flexibility or progressively increases in flexibility distally. In either scenario of the second region, at least one portion of the second region will have a flexibility lower than a lowest flexibility of the first region. For example, if the second region has a uniform flexibility, it may be less flexible than one or all of the distal end, middle, or proximal end of the first region. If the second region progressively decreases in flexibility, one or all of locations within the second region may be less flexible than one or all of the distal end, middle, or proximal end of the first region.

In another example, the catheter may include at least a proximal portion (i.e., a proximal-most portion), a penultimate distal portion (i.e., a second-most distal portion), and a distal portion (i.e., a distal-most portion). Each of the proximal portion, the penultimate distal portion and/or the distal portion may have a uniform stiffness and/or may have a variable stiffness along at least a portion of their respective lengths. The penultimate distal portion may be positioned between the proximal portion and the distal portion. The distal portion may have a greater stiffness than the penultimate distal portion such that a distal tip of the catheter may have a greater stiffness than at least a portion of the penultimate distal portion.

In another example, the catheter may include at least a distal portion, a penultimate distal portion, a penultimate proximal portion (i.e., a second-most proximal portion), and a proximal portion. The stiffness of the distal portion may be greater than the stiffness of the penultimate distal portion. The stiffness of the penultimate proximal portion may be greater than the stiffness of the penultimate distal portion, with the stiffness ramping up along a length of the penultimate proximal portion. The stiffness of the proximal portion may be substantially uniform.

The flexibility/stiffness of a catheter, and particularly a distal region of a catheter, may be determined with one or more of the following features.

In a first example, a stiffness of an outer jacket or sleeve of the catheter may be comprised of regions of different hardness or flexibility. The outer jacket may have a plurality of discrete segments or regions composed of materials of different hardness and/or flexibility. This may be achieved by using different materials for different segments/regions and/or different thicknesses of the different segments/regions. Alternatively, the outer jacket may be formed of a single continuous segment/region that changes in hardness and/or flexibility (e.g., by mixing materials at different areas as the tubular outer jacket is being formed or extruded, or by increasing or decreasing the thickness of the jacket at different areas).

In a second example, a stiffness/flexibility of a catheter may be changed by varying properties of a coiled wire that is located within the catheter walls (e.g., under the outer jacket) and extends at least through a distal region of the catheter. The spacing between the individual coils of the wire can be increased to increase flexibility and decreased to decrease flexibility. Alternatively or additionally, the thickness/diameter of the wire can be increased to decrease flexibility or decreased to decrease flexibility. Hence, the spacing in a coil may initially increase progressively distally and then decrease distally, and/or the diameter of the wire may initially decrease distally and then increase. Alternatively or additionally, the coiled wire may be composed of two or more materials having different flexibility which may be interconnected together (e.g., by welding or the like) so as to vary the flexibility along a length of the coiled wire.

In a third example, a stiffness/flexibility of a catheter may be changed by varying properties of a braided mesh tubular layer that is located within the catheter walls (e.g., between an outer jacket and an inner coil, or underneath an inner coil). The braided mesh may increase its picks-per-inch to decrease flexibility and decrease its picks-per-inch to increase its flexibility. Alternatively or additionally, the wires forming the braided mesh may be increased in thickness to decrease flexibility or decreased in thickness to increase flexibility. Alternatively or additionally, the braided mesh tubular layer may be composed of two or more materials having different flexibility which may be woven together so as to vary the flexibility along a length of the braided mesh tubular layer. Alternatively or additionally, the braided mesh tubular layer may utilize two or more braid patterns having different flexibility so as to vary the flexibility along a length of the braided mesh tubular layer.

In a fourth example, a stiffness/flexibility of a catheter may be changed by varying properties of an inner liner of the catheter that may form the catheter's inner lumen or passage between its proximal and distal end. The inner liner may be formed from two or more tubular portions comprising materials of different hardness or flexibility. In some examples, the liner may be formed from a first tubular portion of a first material (e.g., PTFE) and a second tubular portion connected distal to the first tubular portion and formed from a second material that is softer or more flexible than the first material (e.g., polyolefin elastomer). In another example, a third tubular portion may be connected distal to the second tubular portion and may be composed of the first material or a different third material that is harder or less flexible than the second material. In another example, the liner may not have discrete attached tubular portions and instead may be formed as a single tubular member that gradually changes hardness or flexibility, such as by gradually changing the amount or combination of different materials with different hardness or by changing a thickness of the liner in various areas (e.g., thinner areas have a greater flexibility relative to thicker area).

In a fifth example, a radiopaque marker may extend from a distal end of the catheter to form the distal tip. The radiopaque marker may be integral with the catheter body or may be attached thereto. The radiopaque marker may be comprised of a material that has a greater hardness or lesser flexibility than the immediately adjacent penultimate distal portion of the catheter from which the radiopaque marker extends.

In a sixth example, the distal portion or distal tip may be constructed, in part, using a polymer blend or resin having a radiopaque material such as barium sulfate or tantalum mixed with the polymer. The radiopaque material may be added to the polymer blend or resin prior to or during an extrusion step. This may confer radiopacity properties to the distal portion or distal tip, allowing the user or operator to visualize the catheter tip in-use, but without significantly changing the material properties (e.g., stiffness, hardness) at the distal portion or distal tip of the catheter.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is a side view of a catheter in accordance with an example.

FIG. 2 is a side view of an inner liner of the catheter of FIG. 1 in accordance with an example.

FIG. 3 is a side view of a coil layer of the catheter of FIG. 1 in accordance with an example.

FIG. 4 is a side view of a braided layer of the catheter of FIG. 1 in accordance with an example.

FIG. 5 is a side view of an outer jacket of the catheter of FIG. 1 in accordance with an example.

FIG. 6 is a side view of a catheter hub of the catheter of FIG. 1 in accordance with an example.

FIG. 7 is a perspective cut away view showing a braided layer and an outer jacket layer of the catheter of FIG. 1 in accordance with an example.

FIG. 8 is a stiffness graph of a catheter in accordance with an example.

FIG. 9 is a stiffness graph of a catheter in accordance with an example.

FIG. 10 is a stiffness graph of a catheter in accordance with an example.

FIG. 11 is a stiffness graph of a catheter in accordance with an example.

FIG. 12 is a stiffness graph of a catheter in accordance with an example.

FIG. 13 is a stiffness graph of a catheter in accordance with an example.

FIG. 14 is a diagram illustrating Type I, Type II, and Type III aortic arches.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

While different embodiments and examples may be described in this specification, it is specifically contemplated that any of the features from different embodiments and example can be combined together in any combination. In other words, the features of different embodiments and examples can be mixed and matched with each other. Hence, while every permutation of features from different embodiments and examples may not be explicitly shown, it is the intention of this specification to cover any such combinations.

The terms stiffness, hardness, and flexibility are each used in this specification to describe properties of a catheter. Generally, as stiffness or hardness increases, flexibility decreases. Hence, while one of these terms may be used at different points in this specification, the relationship between the other terms should also be appreciated.

The terms distal and proximal are used in this specification. Distal generally refers to the direction or area towards or near the working end of the catheter (e.g., the end that is advanced to a target location within a patient). Proximal generally refers to the direction or area that remains outside the patient and/or near the physician.

The terms segments, regions, and portions are used throughout this specification and generally refer to or along a certain axial length of the catheter.

The present specification is generally directed to structural features of a catheter. Specifically, structural features that provide desirable flexibility and pushability with reduced kinking.

The structural features and other aspects of this specification may be applied to catheters of any type, size, and/or usage within a human body.

One example type of catheter that may benefit from the structural features and other aspects described in this specification are so called “88” catheters or catheters with an internal lumen passage having a diameter of about 0.088 inch. Currently, such catheters may be one of the larger lumen diameter catheters sold for stroke treatment (e.g., vs 5 French (1.667 mm) and 6 French (2 mm) catheters), and are frequently used as access catheters for clot treatment, removal, or thrombectomy. For some uses, the relatively larger lumen diameter allows a balloon catheter or clot retrieval device to be advanced through the catheter's lumen to a desired location so that the clot can be disrupted, engaged and retrieved into the lumen. Hence, the larger diameter may be helpful in accommodating relatively large medical implants and/or devices such as balloon catheters, clot retrieval devices, or even clots themselves.

These larger 0.088-inch diameter lumens may also be helpful for aspirating clots. The distal tip of the catheter may be advanced near a clot and suction/vacuum may be applied to the catheter's lumen, causing the clot to be sucked into the lumen and removed from the patient's vessel. In addition to the example of 0.088-inch diameter lumens, other sizes greater than 5 French or 6 French may also provide similar advantages and benefits, for example catheters with an internal lumen passage having a 0.092-inch (2.3368 mm) diameter.

The middle cerebral artery (MCA) is the most common artery involved in acute stroke. It branches directly from the internal carotid artery and consists of four main branches, M1, M2, M3, and M4. These vessels provide blood supply to parts of the frontal, temporal, and parietal lobes of the brain, as well as deeper structures including the caudate, internal capsule, and thalamus. Due to their size and location, clots in the M1 and M2 region of the MCA are most commonly treated with clot retrieval devices and/or aspiration, and therefore 0.088-inch lumen catheters can be helpful in providing the best treatment for this area.

To access the brain, physicians typically access the patient's vasculature from their lower extremities (e.g., the femoral artery in the groin area). Radial artery access, for example near the patient's wrist, is also be possible. Additionally, the MCA and vessels approaching it tend to be relatively tortuous. Hence, such a catheter should be relatively long, pushable without kinking, and should retain flexibility to navigate the MCA, which can be difficult to achieve since greater flexibility tends to increase the risk for kinking. Additionally, larger 0.088-inch lumen catheters typically have a more robust and therefore stiffer construction due to their increased size and to prevent its lumen from collapsing during use, in particular while under negative or vacuum pressure during aspiration. Hence, in these larger 0.088-inch lumen catheters, it can be difficult to provide a structure that meets these structural and flexibility needs.

In that regard, the present specification provides several different aspects of catheter construction that may be helpful to achieve this balance of flexibility and pushability in any sized catheter (e.g., 5 French and 6 French), but especially in 0.088 sized catheters.

One aspect of the present specification is directed to a catheter that decreases in stiffness (i.e., increases in flexibility) toward a distal end of the catheter and then increases in stiffness (i.e., decreases in flexibility) further toward the distal end. For example, a distal region of the catheter may generally decrease in stiffness distally but increase in stiffness near or closest to the distal end of the catheter such that a distal portion of the catheter has a greater stiffness than a penultimate distal portion of the catheter immediately adjacent to the distal portion in the proximal direction.

Typically, most catheters have proximal regions that are relatively stiff and, to some degree, have reduced stiffness towards the catheter's distal end. However, by decreasing in stiffness distally and then increasing in stiffness near a distal end of the catheter, certain advantages may be possible in some uses.

For example, the decreased stiffness regions may provide much of the distal region of the catheter with a high degree of flexibility and therefore maneuverability through tortuous vessel pathways while the increased stiffness region closer to the distal end of the catheter may help prevent the lumen of the catheter from collapsing (e.g., under vacuum pressure, when abutting a vessel sidewall turning a tight corner in tortuous vasculature, or upon encountering a region having stenosis), particularly for uses of the catheter involving aspiration through its lumen.

The increased stiffness region may also improve performance when aspirating a clot in some circumstances. For example, as the aspiration through the lumen of the catheter pulls on the clot, the relatively stiffer end region may better cut or dislodge the clot from the vessel. Additionally, the distal end of the catheter may better resist bending or applying suction to non-clot structures (e.g., portions of the vessel). This may be particularly helpful in relatively larger sized catheters, such as 0.088-inch catheters, though other sized catheters may also benefit.

In the case of clot removal in a brain vessel, such as the MCA, the increased stiffness region of a catheter may allow for better navigation through tortuous vessel pathways and branching vessel pathways. While the distal region generally remains relatively flexible, the increased stiffness near an end of the catheter may be beneficial in directing the catheter down a desired branch or twisted vessel region. Put another way, additional increased stiffness may help increase pushability without kinking near a very distal end of the catheter.

The decreasing stiffness (relative to a proximal region of the catheter) and then increasing stiffness along a distal region of the catheter may include different methods of quantifying, describing, or structuring this flexibility. In that regard, it should be noted that a distal region of a catheter may be viewed as having individual, discrete regions or lengths having different stiffness/flexibility relative to adjacent regions, or the distal region may continuously increase and/or decrease in flexibility. Hence, when referencing the flexibility/stiffness of different regions or lengths, such descriptions may include regions of uniform flexibility/stiffness or regions of average flexibility/stiffness.

In one example, a catheter may include a distal region comprising a first region (e.g., length) of the catheter with a single flexibility of a first value and a second region (e.g., length) distal to the first region that has a single flexibility of a second value, lower than the first (i.e., stiffer than the first value).

In one example, a catheter may include a proximal portion, a penultimate distal portion, and a distal portion. The penultimate distal portion may be positioned between the proximal and distal portions. Thus, the penultimate distal portion may be distal with respect to the proximal portion and proximal with respect to the distal portion. The flexibility of each of the proximal, penultimate, and/or distal portions may be uniform along their respective lengths and/or variable along their respective lengths. The flexibility of the distal portion may be less than the flexibility of the penultimate distal portion such that a stiffness of the distal portion is greater than a stiffness of the distal portion.

In another example, a catheter may include a first region (e.g., length of catheter) that progressively increases in flexibility (i.e., decreases in stiffness) distally. In that respect, a proximal end of the first region has a lower flexibility than a distal end of the first region. A second region (e.g., length of catheter) may be located distal of the first region and is either a uniform flexibility or progressively decreases in flexibility. In either scenario of the second region, at least one portion of the second region will have a flexibility lower than a lowest flexibility of the first region. For example, if the second region has a uniform flexibility, it may be less flexible than one or all of the distal end, middle, or proximal end of the first region. If the second region progressively decreases in flexibility, one or all of locations within the second region may be less flexible than one or all of the distal end, middle, or proximal end of the first region.

In any of the prior examples with either the regions of uniform flexibility or progressively increasing/decreasing flexibility, the second region of increased flexibility may extend to the terminal end of the catheter. Alternatively, a further third region may be located distal of the second region and may extend to the terminal end of the catheter. The third region may have an increased, decreased, progressively increasing, progressively decreasing, or the same flexibility relative to the second region (or one or more locations in the second region). The first region in these examples, may have a length within an inclusive range of about 0.5 to 3 cm in length (e.g., 0.5, 1.0, 1.5, 2.0, 2.5, 3.0 cm, or lengths in between these values). In another example, the first region may have a length longer than 3 cm (e.g., up to 15 cm) which may prove useful for peripheral or cardiac applications in addition to the neurological vasculature.

The flexibility/stiffness of a catheter, and particularly a distal region of a catheter, may be determined in a wide variety of manners using a wide variety of features, including but not limited to one or more of the following features.

In a first example, a stiffness of an outer jacket or sleeve of the catheter may be comprised of regions of different hardness (e.g., durometer) or flexibility. The outer jacket may have a plurality of discrete segments or regions composed of materials of different hardness and/or flexibility. This may be achieved by using different materials for different segments/regions and/or different thicknesses of the different segments/regions. Alternatively, the outer jacket may be formed of a single continuous segment/region that changes in hardness and/or flexibility (e.g., by mixing materials at different areas as the tubular outer jacket is being formed or extruded, or by increasing or decreasing the thickness of the jacket at different areas). Alternatively, variable thickness may be achieved by variable rate dip coating.

In a second example, a stiffness/flexibility of a catheter may be changed by varying properties of a coiled wire that is located within the catheter walls (e.g., under the outer jacket) and extends at least through a distal region of the catheter. The spacing between the individual coils of the wire can be increased to increase flexibility and decreased to decrease flexibility. Alternatively or additionally, the thickness/diameter of the wire can be increased to decrease flexibility or decreased to decrease flexibility. Hence, the spacing in a coil may initially progressively increase distally and then decrease distally, and/or the diameter of the wire may initially decrease distally and then increase. Alternatively or additionally, the coiled wire may be composed of two or more materials having different flexibility which may be interconnected together (e.g., by welding or the like) so as to vary the flexibility along a length of the coiled wire. In some embodiments, the material of the coiled wire can be nitinol (e.g., metal alloy of nickel and titanium). In one example, a distal region 118A and/or one or more other regions of the coil layer 118 can be a nitinol material. In this example, by applying a nitinol material along the distal region of the coil wire, the stiffness of the catheter along the distal region may be greater than other regions of the catheter that does not incorporate nitinol material at the respective region of the coil wire. In turn, the increase in stiffness along the distal region of the catheter can result in a reduction of kinking, and the catheter may have improvements in mechanical hysteresis when the catheter is advanced to a target location within a patient. In some embodiments, one or more regions of the coiled wire can be heat-treated to achieve certain mechanical characteristics. On one example, the distal region 118A and/or one or more other regions of the coil layer 118 can be heat-treated to achieve a desired hardness and/or flexibility. In this example, by heat-treating the coiled wire along the distal region of the coiled wire, the stiffness of the catheter along the distal end can increase (relative to other regions that are not heat-treated). Thereby kinking can be reduced, and the catheter may have improvements in mechanical hysteresis.

In a third example, a stiffness/flexibility of a catheter may be changed by varying properties of a braided mesh tubular layer that is located within the catheter walls (e.g., between an outer jacket and an inner coil, or underneath an inner coil). The braided mesh may increase its picks-per-inch to decrease flexibility and decrease its picks-per-inch to increase its flexibility. In one example definition, picks-per-inch is the number of times the wires in a braid cross over each other along the longitudinal axis for each inch of length. Alternatively or additionally, the wires forming the braided mesh may be increased in thickness to decrease flexibility or decreased in thickness to increase flexibility. Alternatively or additionally, the braided mesh tubular layer may be composed of two or more materials having different flexibility which may be woven together so as to vary the flexibility along a length of the braided mesh tubular layer. Alternatively or additionally, the braided mesh tubular layer may utilize two or more braid patterns having different flexibility so as to vary the flexibility along a length of the braided mesh tubular layer. In some embodiments, the material of the braided mesh can be nitinol (e.g., metal alloy of nickel and titanium). In one example, a distal region of the braided mesh and/or other regions of the braided mesh can be a nitinol material, In some embodiments, by incorporating nitinol material along the distal region of the braided mesh, the stiffness of the catheter can be increased. Thereby, kinking can be reduced, and the catheter may have improvements in mechanical hysteresis when the catheter is advanced to a target location within a patient.

In a fourth example, a stiffness/flexibility of a catheter may be changed by varying properties of an inner liner of the catheter that may form the catheter's inner lumen or passage between its proximal and distal end. The inner liner may be formed from two or more tubular portions comprising materials of different hardness or flexibility. In some examples, the liner may be formed from a first tubular portion of a first material (e.g., PTFE) and a second tubular portion connected distal to the first tubular portion and formed from a second material that is softer or more flexible than the first material (e.g., polyolefin elastomer). In another example, a third tubular portion may be connected distal to the second tubular portion and may be composed of the first material or a different third material that is hard or less flexible than the second material. In another example, the liner may not have discrete attached tubular portions and instead may be formed as a single tubular member that gradually changes hardness or flexibility, such as by gradually changing the amount or combination of different materials with different hardness or by changing a thickness of the liner in various areas (e.g., thinner areas have a greater flexibility relative to thicker area).

In a fifth example, a radiopaque marker may extend from a distal end of the catheter to form the distal tip. The radiopaque marker may be integral with the catheter body or may be attached thereto. The radiopaque marker may be comprised of a material that has a greater hardness or lesser flexibility than the immediately adjacent penultimate distal portion of the catheter from which the radiopaque marker extends.

In a sixth example, the distal portion or distal tip may be constructed, in part, using a polymer blend or resin having a radiopaque material such as barium sulfate or tantalum mixed with the polymer. The radiopaque material may be added to the polymer blend or resin prior to or during an extrusion step. This may confer radiopacity properties to the distal portion or distal tip, allowing the user or operator to visualize the catheter tip in-use, but without significantly changing the material properties (e.g., stiffness, hardness) at the distal portion or distal tip of the catheter.

In some examples, the length of the catheter that includes the previously described regions of increased flexibility and decreased flexibility is located within a distal portion of the catheter. The distal portion of the catheter may generally span between the distal end of the catheter and about 10 cm to 40 cm proximally (e.g., 10, 11, 12, 13, 14, 15, 16 17, 18, 19, 20, 21, 22, 23, 24, 25, 26, 27, 28, 29, 30, 31, 32, 33, 34, 35, 36, 37, 38, 39, 40 cm, or lengths in between these values).

Beginning from a proximal end of the distal portion, the distal portion may increase in flexibility distally along its length and then decrease in flexibility. The region of decreased flexibility may be located, in one example, between 0.5 cm to 5 cm from the distal end of the catheter. For example, 0.5, 1, 1.5, 2, 2.5, 3, 3.5, 4, 4.5, 5 cm or lengths in between these values.

In another aspect of the present invention, the catheter stiffness may dramatically increase along a proximal region relative to a distal region. For example, the distal region may be between about 0 to 12 cm relative to the distal end and have an average stiffness of about 5 gf/mm and the proximal region may be between 12 and at least 40 cm and have an average stiffness of 400-600 gf/mm. In some examples, the proximal region may increase in stiffness relative to the distal region by 7000-11000 percent (e.g., 7,000, 7,500, 8,000, 8,500, 9,000, 9,500, 10,000, 10,500, 11,000 percent, and percent values within or near those percentages). These stiffness values may include the previously discussed increase in stiffness at the distal end of the catheter or may not include such a feature (i.e., the stiffness of the distal end may decrease or remain constant).

Again, while these structural features may be applied to catheters of any type, size, and/or usage (e.g., 5 French or 6 French catheters), they may be of particular benefit to catheters having a 0.088-inch lumen diameter size and a working length (i.e., not including a catheter hub) between about 100 cm to 135 cm (e.g., 115 cm or 125 cm). As previously discussed, relatively smaller diameter catheters (e.g., 5 or 6 French) can often remain relatively flexible throughout their length due to their relatively small diameter size without kinking our collapsing their lumens. However, larger diameter catheters, such as 0.088-inch lumen catheters often require greater reinforcement to prevent kinking and/or lumen collapse. This may result in a relatively stiffer catheter overall, which may be undesirable when accessing delicate and tortuous vessels, such as those in the brain (e.g., MCA). In that respect, the structural examples of this specification may be helpful in creating a catheter that has a relatively stiff proximal region (e.g., an average flexibility between about 400 to 600 gf/mm) and a relatively flexible distal region (e.g., an average flexibility between about 350 to 3 gf/mm). This flexibility may be achieved, for example, by the inclusion of one or more of the inner liner, coil layer, braided layer, and outer jacket layer discussed in this specification, including the values of various aspects of each layer, including materials, hardness, coil spacing, wire diameter, braided picks-per-inch, section lengths, and other features discussed further in this specification.

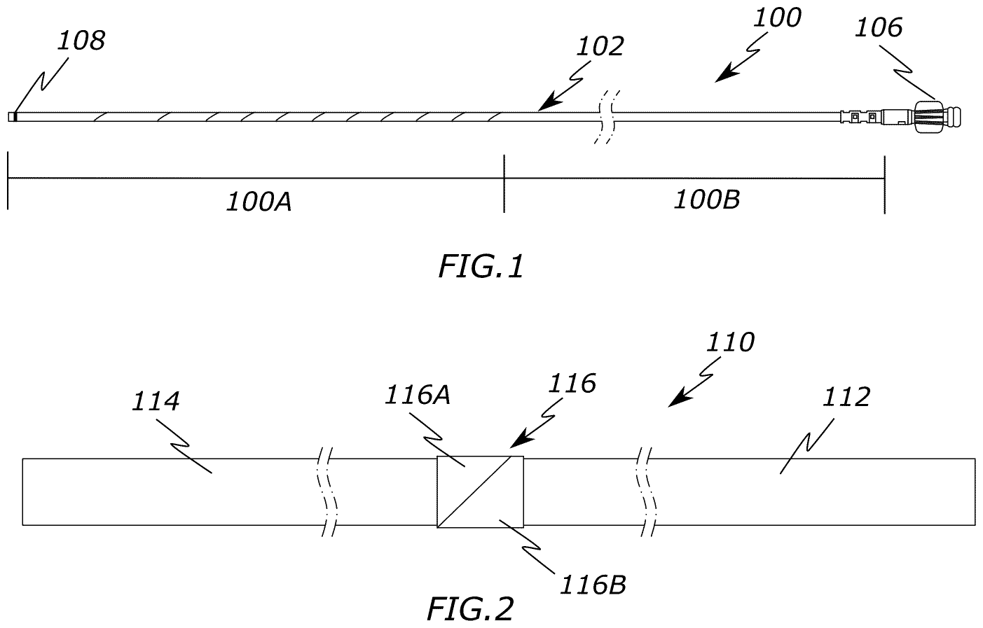

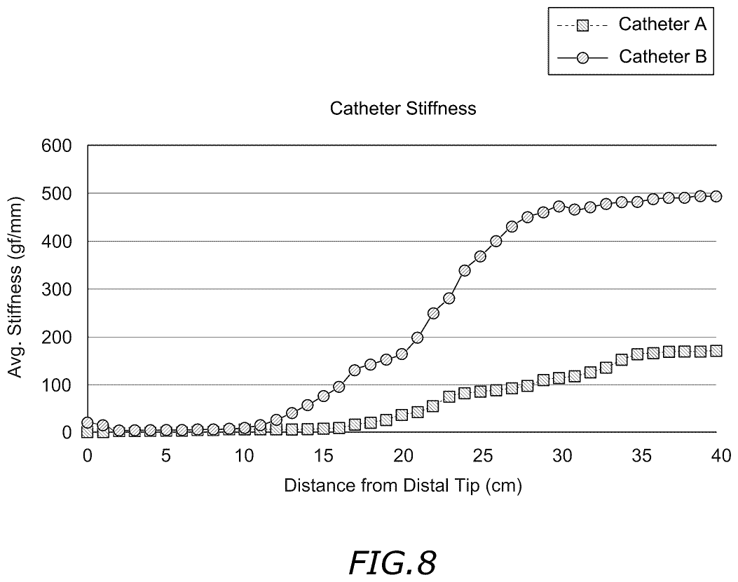

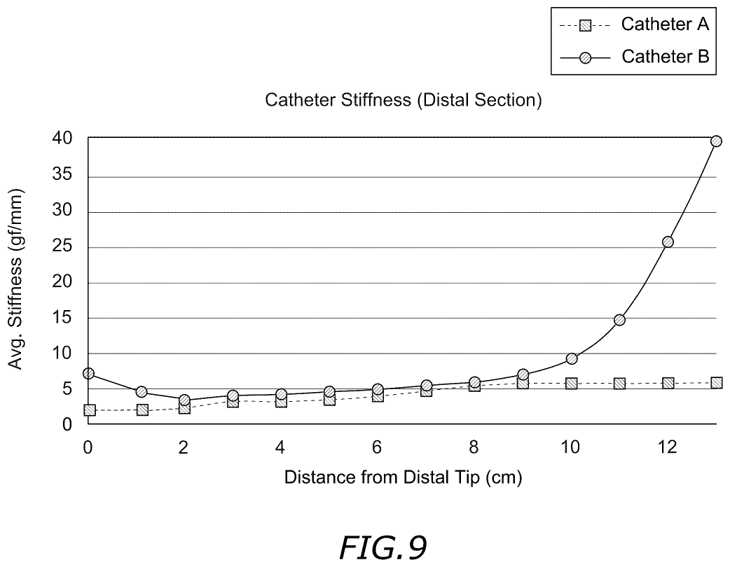

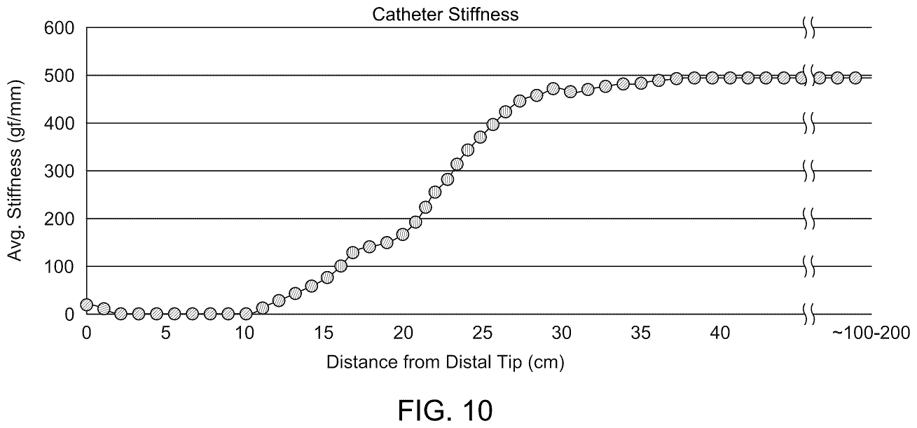

FIGS. 1-7 illustrate various aspects of an example catheter 100 having structural features that provide desirable flexibility and pushability with reduced kinking. An example of this flexibility may be seen in FIGS. 8 and 9, which compare a 0.088-inch lumen diameter catheter 100 (indicated as Catheter B) with a standard 6 French lumen catheter (indicated as Catheter A). Further examples of flexibility may be seen in FIGS. 10-12.

FIG. 10 illustrates that an overall length of the catheter may vary (e.g., between 100 cm and 200 cm, though overall length may in some examples be less than 100 cm or greater than 200 cm) and that a stiffness or flexibility of a proximal portion (e.g., between about 30 cm-40 cm from distal tip to proximal end) may be substantially uniform.

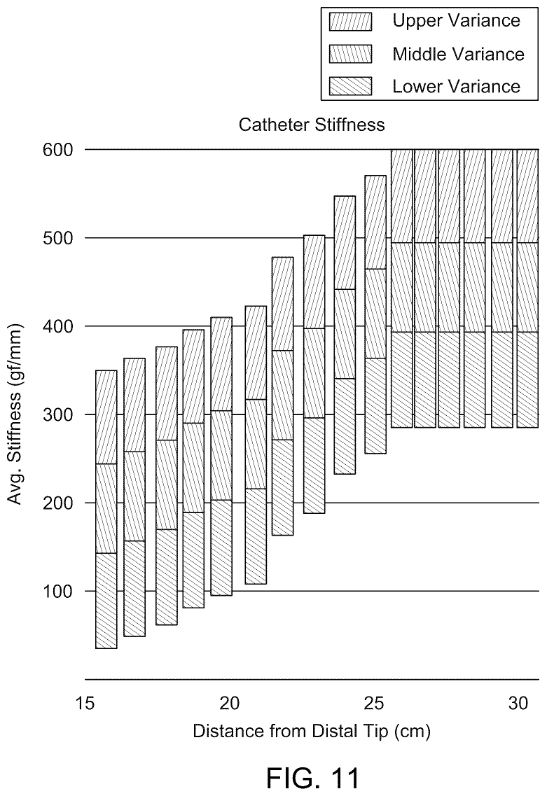

FIG. 11 illustrates non-limiting, example ranges of stiffness values along a portion of the catheter length in which the stiffness ramps up. This portion is illustrated as being between about 15 cm and 30 cm from the distal tip, though it should be appreciated that the positioning of this ramping up portion may vary in different examples and thus should not be construed as limited to the exact values shown in the figure. Further, the effective length of the ramping up portion may also vary and should not be construed as limited by the examples shown in the figures. For example, the ramping up portion may be positioned between about 10 cm and 40 cm from the distal tip, between about 5 cm and 35 cm from the distal tip, between about 15 cm and 30 cm from the distal tip, and the like.

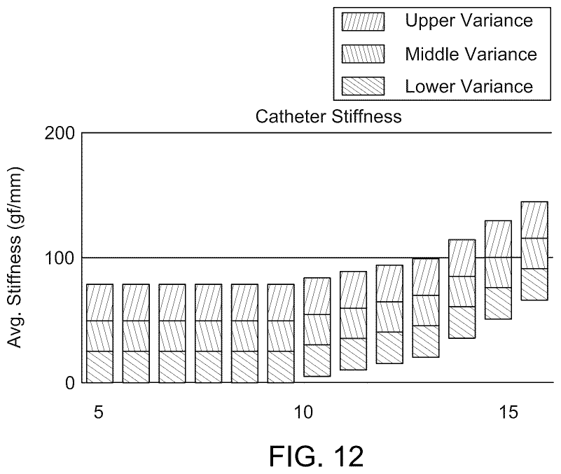

FIG. 12 illustrates non-limiting, example ranges of stiffness values along a penultimate distal portion of the catheter length in which the stiffness may be initially uniform and then start ramping up. This portion is illustrated as being about 5 cm to 15 cm from the distal tip, though it should be appreciated that the positioning of the penultimate distal portion may vary in different examples and thus should not be construed as limited to the exact values shown in the figures. For example, the penultimate distal portion may be considered as starting at 0.5 cm, 1.0 cm, 1.5 cm, 2.0 cm, 2.5 cm, 3.0 cm, 3.5 cm, 4.0 cm, 4.5 cm, or 5.0 cm from the distal tip, depending on the length of the stiffer distal portion. In some examples, such as those in which the length of the stiffer distal portion may be greater than 5 cm, the penultimate distal portion may be considered as starting at a position greater than 5 cm from the distal tip.

Both FIGS. 11-12 illustrate ranges of stiffness measured in gf/mm. More specifically, FIGS. 11-12 illustrate that the respective stiffnesses along the length of the catheter may vary, including portions labeled “upper variance”, “middle variance”, and “lower variance”. Thus, it should be appreciated that the actual values of the stiffness along various segments of the length of the catheter may vary and even may, in some examples, fall outside of the illustrated ranges, which are merely for illustrative purposes.

Note that the preceding data points are merely examples or estimates of values of some configurations according to the present specification and not necessarily of all examples/embodiments.

As seen in these figures, the standard 6 French lumen Catheter A generally remains flexible throughout much of its length, with a proximal portion having an average stiffness around 180 to 190 gf/mm and increasing in flexibility toward its distal end to an average stiffness of 6 to 2 gf/mm. Since the lumen diameter is relatively smaller, it may tend to resist collapse despite being highly flexible.

The example 0.088-inch lumen diameter Catheter B generally remains relatively less flexible throughout much of its length, with a proximal portion having an average stiffness of about 400 to 600 gf/mm or about 500 gf/mm and increasing in flexibility towards its distal end to an average stiffness of about 350 to 2 gf/mm or 5 to 2 gf/mm. This stiffness profile may be achieved with the structural elements discussed further below while also resisting kinking and collapse of the catheter lumen.

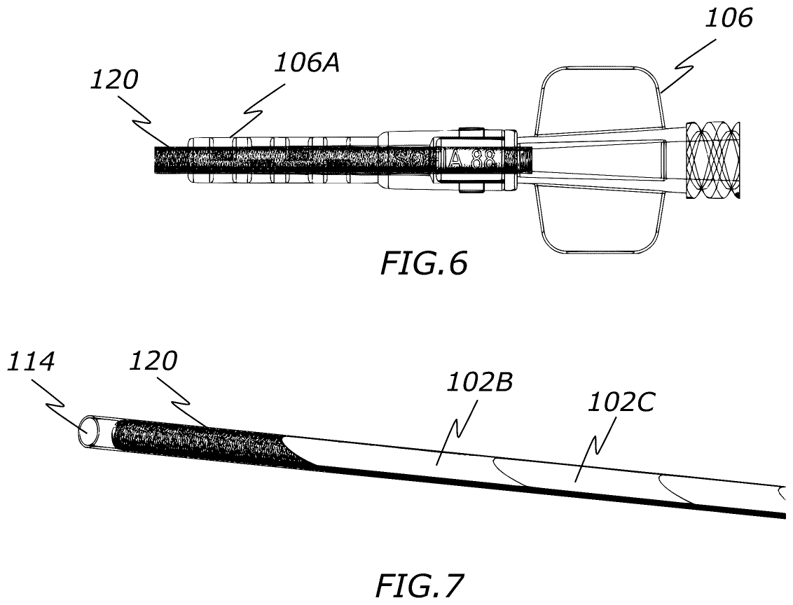

As seen in FIG. 1, the catheter 100 generally may include a working length comprising a distal portion or length 100A and a proximal portion or length 100B. The proximal end of the proximal portion 100B may be connected to a catheter hub 106 and strain relief sleeve 106A (also seen in FIG. 6), and the distal end of the distal portion 100A may optionally include a radiopaque marker 108 located either on the outside or within a wall of the catheter 100. The catheter 100 may include a lumen opening proximally at the catheter hub 106, extending through the proximal portion 100B, distal portion 100A, and opening at the distal end of the catheter 100.

The catheter may at least include several different layers that make up its catheter walls. Specifically, an inner liner 110 (FIG. 2) that may form an inner surface of the catheter's lumen, a coil layer 116 that may be disposed on the inner liner 110, a braided layer 120 that may be disposed on the coil layer 116, and an outer jacket 102 that may be disposed over the braided layer 120. Alternatively, the positions of the coil layer 116 and the braided layer 120 may be swapped or reversed. Alternatively, one of the coil layer 116 or the braided layer 120 may be left out of some examples.

Returning to FIG. 2, the inner liner 110 may include, in one example, a proximal tubular portion 112 and a distal tubular portion 114 that may be either connected together or extruded/molded as a single tube. In the case of using two separate tubes, the distal end of the proximal tubular portion 112 and the proximal end of the distal tubular portion 114 may be connected together in a variety of ways, such as by adhesive, heat/melting, and/or with other components. In the example of FIG. 2, a tie layer 116 in the form a third tube may be placed over both ends. The tie layer 116 may be a single unitary tube or may be composed of a first tube member 116A and a second tube member 1166 that are each bias-cut at reciprocal angles such that their cut surfaces can mate with each other and be connected via adhesive. Adhesive may be further applied between both members 116A, 116B and portions 114, 112, respectively.

The proximal tubular portion 112 may be composed of a material that is relatively stiffer than the distal tubular portion 114. For example, the proximal tubular portion 112 may be composed of PTFE and the distal tubular portion 114 may be composed of a softer polyolefin elastomer. The tie layer may also be composed of a relatively soft material, such as LLDPE (linear low density polyethylene) or LDPE (low density polyethylene).

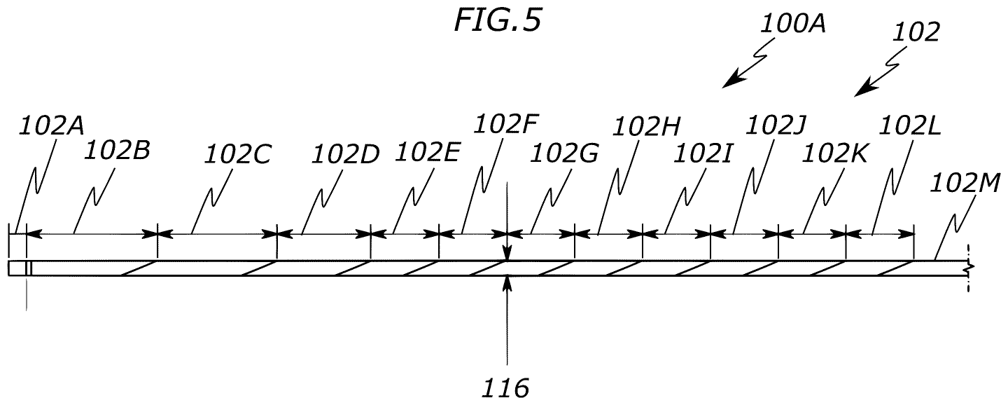

The tie layer 116 and therefore the interface between the two portions 112, 114 may be located within 8 to 20 cm from a distal end of the catheter 100. For example, 8, 9, 10, 11, 12, 13, 14, 15, 16, 17, 18, 19, 20 cm, or positions between those values. FIG. 5 illustrates one example location of the tie layer 116 relative to a portion of the outer jacket 102 in the distal portion 100A.

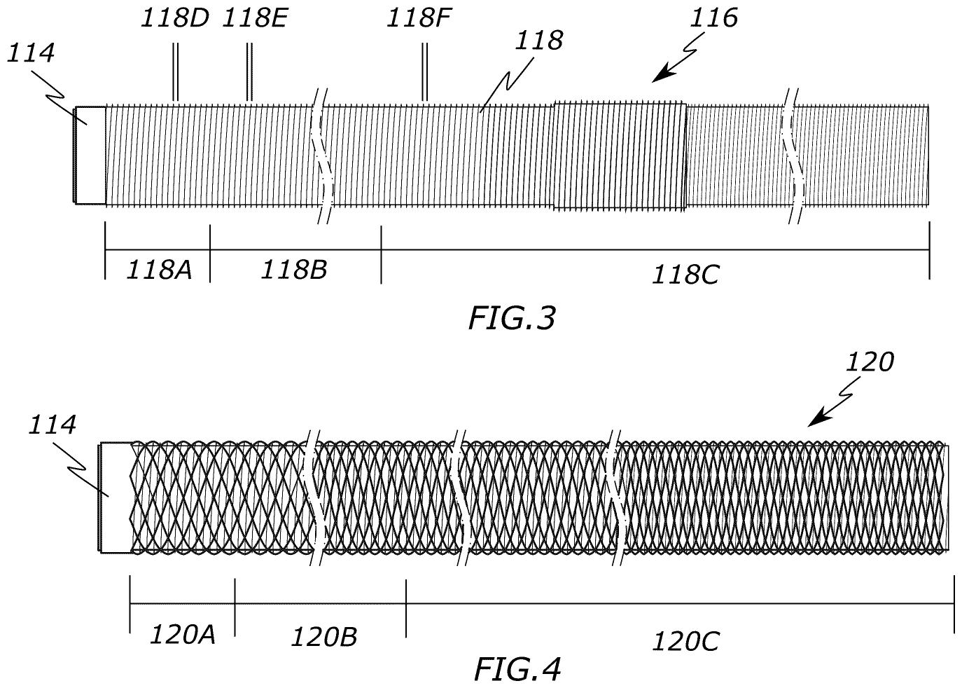

The coil layer 118 can be seen in FIG. 3 and may be positioned over the inner liner 110, as seen by the location of the tie layer 116. The coil layer 118 may extend to the very distal end of the catheter 100 or may terminate a short distance before the distal end of the catheter 100 (e.g., 0.5, 1.0, 1.5, or 2.0 cm from the distal end). The coil layer 118 may be composed of a coiled wire that varies in pitch in different regions or lengths. Generally, the coil layer 118 increases in flexibility distally, however, the coil layer 118 may optionally decrease in flexibility near its distal end.

In one example, the coil layer 118 may have a distal region 118A, an intermediate region 118B, and a proximal region 118C. The distal region 118A may have a length 118D within an inclusive range of 1.5 cm and 3 cm (e.g., 1.5, 1.6, 1.7, 1.8, 1.9, 2.0, 2.1, 2.2, 2.3, 2.4, 2.5, 2.6, 2.7, 2.8, 2.9, 3.0 cm, or lengths in between those values).

The intermediate region 118B may have a length 118E within an inclusive range of 7 cm to 10 cm (e.g., 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9.0 cm, or lengths in between those values).

The distal region 118C may have a length 118F within an inclusive range of 95 to 120 cm (e.g., 95, 96, 97, 98, 99, 100, 101, 102, 103, 104, 105, 106, 107, 108, 109, 110, 111, 112, 113, 114, 115, 116, 117, 118, 119, 120 cm, or lengths in between those values).

The flexibility of the coil layer 118 may be controlled by the spacing of individual coils of the layer (e.g., a larger spacing increases flexibility), by changing the diameter of the wire (e.g., a smaller diameter increases flexibility), and/or by using a material of certain softness. In one example, the coil layer 118 is formed from a Nitinol wire which is relatively soft, or a stainless-steel wire which is relatively stiffer than the Nitinol wire.

In one example, the intermediate region 118B has greater flexibility than the proximal region 118C, and the distal region 118A has greater flexibility than the intermediate region 118B. In such an example, the individual loops of the proximal region 118C may be spaced within an inclusive range of about 0.003 inch to 0.0055 inch from each other (e.g., 0.003, 0.0035, 0.004, 0.0045, 0.005, 0.0055 inch, or lengths in between those values). Individual loops of the intermediate region 118B may be spaced within an inclusive range of about 0.005 inch to 0.007 inch (e.g., 0.005, 0.0055, 0.006, 0.0065, 0.007, 0.0075, 0.008 inch or lengths in between those values). Individual loops of the distal region 118A may be spaced within an inclusive range of about 0.006 inch to 0.010 inch (e.g., 0.006, 0.0065, 0.007, 0.0075, 0.008, 0.009, 0.0095, 0.001 inch, or lengths in between those values).

In another example, the intermediate region 118B has greater flexibility than the proximal region 118C, and the distal region 118A has greater flexibility than the intermediate region 118B. In such an example, the individual loops of the proximal region 118C may be spaced within an inclusive range of about 0.003 inch to 0.0055 inch from each other (e.g., 0.003, 0.0035, 0.004, 0.0045, 0.005, 0.0055 inch, or lengths in between those values). Individual loops of the intermediate region 118B may be spaced within an inclusive range of about 0.005 inch to 0.007 inch (e.g., 0.005, 0.0055, 0.006, 0.0065, 0.007, 0.0075, 0.008 inch or lengths in between those values). Individual loops of the distal region 118A may be spaced within an inclusive range of about 0.003 inch to 0.0055 inch from each other (e.g., 0.003, 0.0035, 0.004, 0.0045, 0.005, 0.0055 inch, or lengths in between those values).

The braided layer 120 can be seen best in FIG. 4 and may be positioned over the coil layer 110 or alternatively underneath the coil layer 110. The braided layer 120 may extend to the very distal end of the catheter 100 or may terminate a short distance before the distal end of the catheter 100 (e.g., 0.5, 1.0, 1.5, or 2.0 cm from the distal end), as well as may extend to the beginning of the catheter hub 106 (FIG. 6). The braided layer 120 may be composed of one or more wires that are braid together. Generally, the braided layer 120 increases in flexibility distally, however, the braided layer 118 may optionally also decrease in flexibility near its distal end.

In one example, the braided layer 120 may have a distal region 120A, an intermediate region 118B, and a proximal region 118C. The distal region 120A may have a length within an inclusive range of 9 cm to 12 cm (e.g., 9, 9.5, 10, 10.5, 11, 11.5, 12 cm, or lengths in between those values). The intermediate region 120B may have a length within an inclusive range of 1.5 cm and 3 cm (e.g., 1.5, 1.6, 1.7, 1.8, 1.9, 2.0, 2.1, 2.2, 2.3, 2.4, 2.5, 2.6, 2.7, 2.8, 2.9, 3.0 cm, or lengths in between those values). Optionally, both the distal region 120A and the intermediate region 120B may have a length about the same as the distal region 118A and intermediate region 118B of the coil layer 118 (e.g., about 10.5 cm combined length).

The distal region 120C may have a length within an inclusive range of 95 to 120 cm (e.g., 95, 96, 97, 98, 99, 100, 101, 102, 103, 104, 105, 106, 107, 108, 109, 110, 111, 112, 113, 114, 115, 116, 117, 118, 119, 120 cm, or lengths in between those values).

The flexibility of the braided layer 120 may be controller by the density of the braid or the braids picks-per-inch (higher picks-per-inch reduces flexibility), by changing the diameter of the wire (e.g., a smaller diameter increases flexibility), and/or by using a material of a certain softness. In one example, the braided layer 120 is formed from one or a plurality of braided Nitinol wires which are relatively soft, or one or a plurality of stainless-steel wires which are relatively stiffer than the Nitinol wires. In one example, a stiffer braided stainless steel wire region can be fixed to a more flexible Nitinol wire region.

In one example, the intermediate region 120B has greater flexibility than the proximal region 120C, and the distal region 120A has greater flexibility than the intermediate region 120B. In such an example, the picks-per-inch of the proximal region 120C may be within an inclusive range of 120 PPI to 140 PPI (e.g., 120, 121, 122, 123, 124, 125, 126, 127, 128, 129, 130, 131, 132, 133, 134, 135, 136, 137, 138, 139, 140 PPI, or lengths in between those values).

The picks-per-inch of the distal region 120A may be with an inclusive range of about 65 PPI to 90 PPI (e.g., 65, 66, 67, 68, 69, 70, 71, 72, 73, 74, 75, 76, 77, 78, 79, 80, 81, 82, 83, 84, 85, 86, 87, 88, 89, 90 PPI, or lengths in between those values).

The picks-per-inch of the intermediate region 120B may transition between the picks-per-inch of the proximal region 120C to the picks-per-inch of the distal region 120A.

In another example, the intermediate region 120B has greater flexibility than the proximal region 120C, and the distal region 120A has greater flexibility than the intermediate region 120B. In such an example, the picks-per-inch of the proximal region 120C and the distal region 120A may be within an inclusive range of 120 PPI to 140 PPI (e.g., 120, 121, 122, 123, 124, 125, 126, 127, 128, 129, 130, 131, 132, 133, 134, 135, 136, 137, 138, 139, 140 PPI, or lengths in between those values). The picks-per-inch of the intermediate region 120B may be with an inclusive range of about 65 PPI to 90 PPI (e.g., 65, 66, 67, 68, 69, 70, 71, 72, 73, 74, 75, 76, 77, 78, 79, 80, 81, 82, 83, 84, 85, 86, 87, 88, 89, 90 PPI, or lengths in between those values).

The outer jacket 102 can be seen in FIGS. 1 and 7 and a portion of the outer jacket 102 within the distal portion 100A of the catheter 100 can be seen in FIG. 5. The outer jacket 102 may extend to the very distal end of the catheter 100 and to the catheter hub 106 at a proximal end of the catheter 100. In one example, the outer jacket 102 may have a length within an inclusive range of about 110 cm to 135 cm (e.g., having a working length of about 115 or 125). Generally, the flexibility or hardness (e.g., durometer) of the outer jacket 102 increases in flexibility or decreases in hardness towards a distal end of the catheter 100, however, the outer jacket 102 may optionally also decrease in flexibility or increase in hardness near the distal end of the catheter 100 (e.g., within a length of 0.5 to 3 cm of the distal end).

The outer jacket 102 is further described in terms of discrete jacket segments. However, such discussion should be understood to both encompass tubular segments of different characteristics that are connected to each other to form a tubular layer, or a unitary tubular structure that was molded or extruded as a single tube with gradually changing materials and/or thicknesses along its length. In that respect, the tubular segments may abruptly change in characteristics (e.g., hardness) or may more gradually change in characteristics along the outer jacket's length. In the example of discrete, attached segments, the segments my have an axially perpendicular cut (e.g., between segment 102A and 102B) or may have a biased angle cut relative to the catheter's axis (e.g., 45 degrees) that helps create a smoother transition between materials of a different durometer. In the example of biased cut segments, length measurements can be between each end tip of the segment, between only portions with no bias cut, or from one bias cut tip to location just prior to the opposite bias cut tip (such as set out in FIG. 5). The segments are also described in terms of hardness and durometer. However, the increased and decreased sections of different durometer should also be understood to have similar increased or decreased flexibility. All durometer values in this specification are Shore A values.

A proximal segment 102M of the outer jacket 102 may generally have the least flexibility of the outer jacket 102 so as to provide maximize the support and stiffness. In one example, the proximal segment 102M can be composed of a relatively hard material, such as Pebax and Nylon (Grilamid L25). The proximal segment 102M may have a wall thickness within an inclusive range of 0.004 to 0.007 inch (e.g., 0.004, 0.005, 0.006, 0.007, and thicknesses in between those values). The proximal segment 102M may have a length within an inclusive range of about 80 cm to about 100 cm (e.g., 80, 81, 82, 83, 84, 85, 86, 87, 88, 89, 90, 91, 92, 93, 94, 95, 96, 97, 98, 99, 100 cm and lengths in between these values). The hardness can be in a range of 50 A to 120 A durometer.

The distal segments 102A-102L may have different flexibilities from each other relative to proximally and distally adjacent segments. In one example, this can be achieved by using different materials for each segment and/or different thicknesses of each segment. For example, the distal segments 102A-102L may be composed of thermoplastic polyurethane elastomer (TPU) with different hardness or durometer. In one example, the distal segments 102A-102L all have the same thickness within an inclusive range of 0.002 to 0.010 inch and different durometers relative to proximally or distally adjacent segments to achieve changes flexibility.

In one example, the distal segments 102 generally each have increased flexibility and/or a decreased durometer relative to an immediately proximal segment. In another example, the distal segments 102 generally each have increased flexibility and/or a decreased durometer relative to an immediately proximal segment, except for one or more segments at the very distal end of the outer jacket 102.

In one example, only distal segment 102A decreases in flexibility or increases in durometer relative to distal segment 102B. Distal segment 102A may have a length within an inclusive range of 0.5 cm to 3.0 cm (e.g., 0.5, 1.0, 1.5, 2.0, 2.5, 3.0 cm, or lengths in between these values). The distal segment 102A may be composed of a TPU or similar material with a durometer within a range of about 20 A to 100 A (e.g., 20 A, 21 A, 22 A, 23 A, 24 A, 25 A, 26 A, 27 A, 28 A, 29 A, 30 A, 31 A, 32 A, 33 A, 34 A, 35 A, 36 A, 37 A, 38 A, 39 A, 40 A, 41 A, 42 A, 43 A, 44 A, 45 A, 46 A, 47 A, 48 A, 49 A, 50 A, 51 A, 52 A, 53 A, 54 A, 55 A, 56 A, 57 A, 58 A, 59 A, 60 A, 61 A, 62 A, 63 A, 64 A, 65 A, 66 A, 67 A, 68 A, 69 A, 70 A, 71 A, 72 A, 73 A, 74 A, 75 A, 76 A, 77 A, 78 A, 79 A, 80 A, 81 A, 82 A, 83 A, 84 A, 85 A, 86 A, 87 A, 88 A, 89 A, 90 A, 91 A, 92 A, 93 A, 94 A, 95 A, 96 A, 97 A, 98 A, 99 A, 100 A and durometers in between these values). The distal segment 102B may be composed of a TPU or similar material with a durometer that is at least one value lower than that of the distal segment 102A, but as many as 20 to 30 units lower. For example, the distal segment 102A may be 27 A-32 A (e.g., 27 A, 28 A, 29 A, 30 A, 31 A, or 32 A) and the distal segment 102B may be 10 A-14 A (e.g., 10 A, 11 A, 12 A, 13 A, or 14 A).

In one example, only distal segments 102A and 102B decrease in flexibility or increases in durometer relative to distal segment 102B. Distal segments 102A and 102B may have a length within an inclusive range of 0.5 cm to 3.0 cm (e.g., 0.5, 1.0, 1.5, 2.0, 2.5, 3.0 cm, or lengths in between these values). The distal segment 102C may be composed of a TPU or similar material with a durometer that is at least one unit lower than that of the distal segment 102B, but as many as 20 to 30 units lower. The distal segment 102A may be the same or at least one unit lower than the distal segment 102B. For example, the distal segment 102A may have a durometer within an inclusive range of 27 A-32 A (e.g., 27 A, 28 A, 29 A, 30 A, 31 A, 32 A, or durometers in between these values) and the distal segment 102B may have a durometer the same or lower than segment 102A within the same inclusive range, while distal segment 102C may have a durometer within an inclusive range of about 10 A-14 A (e.g., 10 A, 11 A, 12 A, 13 A, 14 A, or durometers in between these values). This pattern may be further expanded such that any number of several distal segments 102A, 102B, 102, 102D, and/or 102E may progressively increase in durometer and decrease in flexibility in a proximal direction with durometer values within an inclusive range of 27 A-32 A (e.g., 27 A, 28 A, 29 A, 30 A, 31 A, 32 A, or durometers in between these values), so long as each section is the same or of higher durometer than a proximally adjacent section.

The distal segment 102A is described as a segment of the outer jacket 102. However, the distal segment may alternately be part of a different layer and extend past the segment 102B of the outer jacket 102. For example, the distal segment 102A may be attached to or may be a portion of the inner liner 110 (e.g., portion 114).

Note, sections 102L through 102F may also generally incrementally decrease in hardness (increase in flexibility) towards a distal direction, though specific example Shore values may not be necessarily be provided in this specification. For example, these Shore values may be within an inclusive range of about 20 D to 100 D.

Several example durometer progressions are provided in Table 1 below. While the increase in durometer at a distal portion (e.g., 102A) is one aspect of the outer jacket 102, another aspect is the progression of durometer increase in proximal sections, as seen below, which may dramatically increase stiffness proximally. This dramatic increase in proximal stiffness may particularly help with relatively larger catheters, as defined elsewhere in the this specification, to provide pushability without kinking or collapsing, yet allowing navigation of tortuous vessels with its distal portion.

TABLE 1

| | Example 1 | Example 2 | Example 3 |
|---|---|---|---|
| Distal | (Durometer, | (Durometer, | (Durometer, |
| Section | Shore) | Shore) | Shore) |
| 102A | 30A | 30A | 30A |
| 102B | 12A | 28A | 30A |
| 102C | 18A | 12A | 12A |
| 102D | 30A | 18A | 18A |
| 102E | 45A | 30A | 30A |
| 102F | 60A | 45A | 45A |
| 102G | 70A | 60A | 60A |
| 102H | 35D | 35D | 35D |
| 102I | 45D | 45D | 45D |
| 102J | 55D | 55D | 55D |
| 102K | 63D | 63D | 63D |
| 102L | 72D | 72D | 72D |

Another approach to describing the outer jacket 102 within the distal portion 100A of the catheter 100 is that at certain lengths from the distal end of the catheter 100, the outer jacket 102 may have a certain hardness/durometer or average hardness/durometer. Example lengths and durometers are provided in Table 2 below. The lengths below may be referred to as distal most (0-0.5 cm or 0-0.05 cm), second distal most (0.5-3.5 cm or 0.05-3.5 cm), and similarly for the remaining segments. The use of a relatively shorter distal-most section (e.g. 0-0.5 cm in length) may assist in navigating the aortic arch.

TABLE 2

| Length from | Example 1 | Example 2 | Example 3 |
|---|---|---|---|
| Distal End (cm) | (Durometer) | (Durometer) | (Durometer) |
| 0-0.5 | 30A | 30A | 30A |
| 0.5-3.5 | 12A | 28A | 30A |
| 3.5-6.6 | 18A | 12A | 12A |
| 6.5-9.0 | 30A | 18A | 18A |
| 9.0-11.0 | 45A | 30A | 30A |
| 11.0-13.0 | 60A | 45A | 45A |
| 13.0-15.0 | 70A | 60A | 60A |

TABLE 2B

| Length from | Example 1 | Example 2 | Example 3 |
|---|---|---|---|
| Distal End (cm) | (Durometer) | (Durometer) | (Durometer) |
| 0-0.05 | 30A | 30A | 30A |
| 0.05-3.5 | 12A | 28A | 30A |
| 3.5-6.6 | 18A | 12A | 12A |
| 6.5-9.0 | 30A | 18A | 18A |
| 9.0-11.0 | 45A | 30A | 30A |
| 11.0-13.0 | 60A | 45A | 45A |
| 13.0-15.0 | 70A | 60A | 60A |

TABLE 2C

| Length from | Example 1 | Example 2 | Example 3 |
|---|---|---|---|
| Distal End (cm) | (Durometer) | (Durometer) | (Durometer) |
| 0-0.05 | 30A | 30A | 30A |
| 0.05-3.5 | 18A | 18A | 18A |
| 3.5-6.6 | 18A | 12A | 12A |
| 6.5-9.0 | 30A | 18A | 18A |
| 9.0-11.0 | 45A | 30A | 30A |
| 11.0-13.0 | 60A | 45A | 45A |
| 13.0-15.0 | 70A | 60A | 60A |

In the examples shown above in Table 2B and Table 2C, the distal-most section having a durometer of 30 A Shore is 0-0.05 cm in length. Alternatively, this section may range from 0-0.01 cm in length to 0-0.1 cm in length (or up to 0.5 cm in length, as shown in Table 2). This may be advantageous or beneficial to the operator when navigating the aortic arch (shown in FIG. 14) and in particular, Type III aortic arches. Type III aortic arches are relatively uncommon in the general population. Estimates vary, but it is likely under 25% of the adult population. However, Type III aortic arches are more common amongst elderly patients and patients with risk factors for strokes, aneurysms, and other diseases or acute medical issues that may require intravascular intervention. Again, estimates vary, but the prevalence of Type III aortic arches in patients undergoing neuro-endovascular procedures may be as high as 50% or more. Type III aortic arches are particularly challenging to navigate during endovascular procedures due to the dual hairpin turn between descending aorta, the aortic arch, and the brachiocephalic artery (access to the right common carotid artery (RCA)) or left common carotid artery (LCA). A catheter such as those described herein, and in particular catheters having the a higher durometer in the distal-most section (e.g., as described in Tables 2, 2B, 2C), may be advantageous or beneficial to the operator and/or patient by providing a distal tip that is stiff enough so as not to collapse or “accordion fold” when the distal tip contacts the blood vessel wall when navigating tortuous anatomy (e.g., in a Type III aortic arch). However, the distal catheter section immediately proximal to the distal tip (i.e., the penultimate section) may be soft enough to provide sufficient flexibility near the distal end of the catheter. This may provide improved navigability and trackability in challenging and/or tortuous anatomies such as at least a Type II or Type III aortic arch.

Another approach to describing the outer jacket 102 within the distal portion 100A of the catheter 100 is that a hardness or durometer of the outer jacket 102 may roughly double, triple, or similarly increase from a proximal section to a distally adjacent distal section (e.g., from distal section 102B to distal section 102A). For example, the durometer between distal section 102B to 102A may increase by about 100 (i.e., double), 110, 120, 130, 140, 150, 160, 170, 180, 190, 200, 210, 220, 230, 240, 250, 260, 270, 280, 290, 300 (i.e., triple), 310, 320, 330, 340, 350 percent or percentages in between those values. In such a scenario, the Another approach to describing the distal portion 100A of the catheter 100 as a whole is that a stiffness may roughly double, triple, or similarly increase from a proximal point of the distal portion 100A to a distal point fo the distal portion 100A. For example, a point within 0 to 2 cm from the distal end may roughly double, triple, or similarly increased over a point within 2 cm to 4 cm. For example, the stiffness between the two points may increase by about 100 (i.e., double), 110, 120, 130, 140, 150, 160, 170, 180, 190, 200, 210, 220, 230, 240, 250, 260, 270, 280, 290, 300 (i.e., triple), 310, 320, 330, 340, 350 percent or percentages in between those values.

Another approach to describing the flexibility of the catheter as a whole may include flexibility values at different locations from a distal end of the catheter 100. Example values are shown in Tables 3 and 4 below. These may be example values inclusive of ranges of 10% above or below each of these values or may be viewed as average values for a length range overlapping a specified length value (e.g., equal length overlap proximally and distally).

TABLE 3

| Length from | Example 1 | Example 2 | Example 3 |
|---|---|---|---|
| Distal End (cm) | (gf/mm) | (gf/mm) | (gf/mm) |
| 0.5 | 7 | 8 | 7 |
| 1 | 5 | 4 | 7 |
| 2 | 3.5 | 3 | 3.5 |
| 3 | 4 | 4 | 4 |
| 4 | 4.5 | 4.5 | 4.5 |
| 6 | 5 | 5 | 5 |
| 8 | 6 | 6 | 6 |
| 10 | 9 | 9 | 9 |
| 12 | 26 | 26 | 26 |
| 13 | 40 | 40 | 40 |

TABLE 4

| Length from | Example 1 | Example 2 | Example 3 |
|---|---|---|---|
| Distal End (cm) | (gf/mm) | (gf/mm) | (gf/mm) |
| 0.5 | 1 | 3 | 1 |
| 1 | 3 | 3 | 2 |
| 2 | 3 | 4 | 3 |
| 3 | 4 | 5 | 4 |
| 4 | 4.5 | 4.5 | 4.5 |
| 6 | 5 | 5 | 5 |
| 8 | 6 | 6 | 6 |
| 10 | 9 | 9 | 9 |
| 12 | 26 | 26 | 26 |
| 13 | 40 | 40 | 40 |

The following examples refer to example combinations of the previously described catheter layers. While specific numbers (e.g., lengths, PPI, coil widths, durometer, etc.) may not be specified below, reference is made to the example numbers set for in the prior discussion of each of these layers. The 0.5 cm distal location may have a stiffness within an inclusive range between 4 gf/mm and 20 gf/mm. The 2 cm distal location may have a stiffness within an inclusive range between 2 gf/mm to 18 gf/mm.

In one example, the catheter 100 includes a coil layer 118 that increases in flexibility until its distal end, a braided layer 120 that increases in flexibility until its distal end, and an outer jacket layer 102 that increases in flexibility towards its distal end, but then decreases in flexibility until its distal end (e.g., decreases between a length of 0.5 to 3 cm from its distal end).

In one example, the catheter 100 includes a coil layer 118 that increases in flexibility until its distal end but then decreases in flexibility until its distal end (e.g., decreases between a length of 0.5 to 3 cm from its distal end), a braided layer 120 that increases in flexibility until its distal end, and an outer jacket layer 102 that increases in flexibility towards its distal end.

In one example, the catheter 100 includes a coil layer 118 that increases in flexibility until its distal end, a braided layer 120 that increases in flexibility until its distal end but then decreases in flexibility until its distal end (e.g., decreases between a length of 0.5 to 3 cm from its distal end), and an outer jacket layer 102 that increases in flexibility towards its distal end.

In one example, the catheter 100 includes a coil layer 118 that increases in flexibility until its distal end but then decreases in flexibility until its distal end (e.g., decreases between a length of 0.5 to 3 cm from its distal end), a braided layer 120 that increases in flexibility until its distal end but then decreases in flexibility until its distal end (e.g., decreases between a length of 0.5 to 3 cm from its distal end), and an outer jacket layer 102 that increases in flexibility towards its distal end but then decreases in flexibility until its distal end (e.g., decreases between a length of 0.5 to 3 cm from its distal end).

In another example, without regard to the stiffness of the very distal tip of the catheter 100, the proximal portions of the catheter 100 may have a catheter flexibility profile with a relatively high proximal stiffness and relatively low distal stiffness (for example, as seen in FIGS. 8 and 9 between 2-40 cm from the distal tip of the catheter). This combination of high proximal stiffness and low distal stiffness may be particularly effective for navigating and resisting kinks and collapse for relatively larger catheters.

For example, between the distal tip and a location about 7-12 cm proximal, the catheter 100 may have an average stiffness of about 5 gf/mm. Between a location about 7-12 cm proximal and a location about 40-100 cm from the distal tip, the average stiffness may be about 450-550 gf/mm. Put another way, between 7-12 cm proximal the distal tip and a location about 40-100 cm from the distal tip has an average stiffness that is about 7000-11000 percent higher than between the distal tip and a location about 7-12 cm proximal, and more specifically 9000-10000 percent higher (e.g., 7,000, 7,500, 8,000, 8,500, 9,000, 9,500, 10,000, 10,500, 11,000 percent, and percent values within or near those percentages).

Described another way, between a distal end of the catheter and about 10 cm proximal of the distal end, an average stiffness is between about 2-10 gf/mm, between about 10 cm and 13 cm from the distal tip, the average stiffness proximally increases between about 9 gf/mm to 39 gf/mm, between about 13 cm and 30 cm from the distal tip, the average stiffness proximally increases between about 29 gf/mm to 480, and between about 30 cm and 40 cm from the distal tip, the average stiffness proximally increases between about 480 gf/mm to 495 gf/mm. These example values, and the further specific example values in Table 5 may vary by about 10 percent.

TABLE 5

| Length from | Example 1 | Example 2 | Example 3 |
|---|---|---|---|
| Distal End (cm) | (gf/mm) | (gf/mm) | (gf/mm) |
| 0-2 | 1 | 3 | 7 |
| 3 | 4 | 3 | 2 |
| 4 | 4 | 4 | 3 |
| 6 | 5 | 5 | 5 |
| 8 | 6 | 6 | 6 |
| 10 | 9 | 9 | 9 |
| 12 | 26 | 26 | 26 |
| 13 | 39 | 39 | 39 |
| 15 | 90 | 90 | 90 |
| 20 | 180 | 180 | 180 |
| 25 | 400 | 400 | 400 |
| 30 | 480 | 480 | 480 |
| 35 | 490 | 490 | 490 |
| 40 | 495 | 495 | 495 |

As best illustrated in FIGS. 8-13, an example catheter may comprise varying stiffness profiles along its length. The catheter may include at least a distal portion, a penultimate distal portion, a penultimate proximal portion, and a proximal portion. The stiffness of the distal portion may be greater than the stiffness of the penultimate distal portion. The stiffness of the penultimate proximal portion may be greater than the stiffness of the penultimate distal portion, with the stiffness ramping up along a length of the penultimate proximal portion. The stiffness of the proximal portion may be substantially uniform.

As an example, a distal-most portion of the catheter may comprise a stiffness that is greater than a stiffness of a penultimate distal portion of the catheter proximally adjacent to the distal-most portion. The penultimate distal portion may comprise a substantially uniform, lower stiffness, before transitioning to a penultimate proximal portion having a ramped up stiffness. A proximal portion of the catheter may comprise a substantially uniform stiffness.

As an example, the catheter may comprise an elongated catheter body and an outer jacket located along the elongated catheter body having a distal region. The distal region may have a variable hardness that, in a distal direction along a longitudinal axis, decreases in hardness toward a distal end of the catheter body and then increases in hardness until the distal-most end of the catheter body. The hardness of the outer jacket at the distal-most end of the catheter body may be between 20 A and 50 A Shore. The hardness of the outer jacket at a portion of the outer jacket proximal to the distal-most end of the catheter body may be between 10 A and 20 A Shore. The distal region having the variable hardness decreasing in hardness toward a distal end of the catheter body may comprise a first outer jacket segment having a first hardness bonded to an interface region to a second outer jacket segment having a second hardness. The interface region may have a smooth transition from the first hardness to the second hardness. The interface region may have a length of at least 0.5 cm, or between 0.1 cm and 1.0 cm. The interface region may comprise a bonding (e.g., a butt bonding or a scarf bonding) between two adjacent regions having different hardnesses or durometers. The bonding may be a diagonal bonding (e.g., a scarf bonding), a linear bonding (e.g., a butt bonding), or various other types of bondings. Advantages of a diagonal or scarf bonding may include at least that transition between the respective stiffnesses or hardnesses of each of the adjacent, bonded regions may be smoother than with other types of bonding (e.g., butt bonding), which would have a sharper transition zone between the respective regions.

As an example, a catheter may comprise an elongated catheter body having a distal portion, a proximal portion, and a medial portion therebetween. The distal portion may have a first stiffness that is less than 50 gf/mm, the medial portion may have a second stiffness, and the proximal portion may have a third stiffness that is greater than 400 gf/mm. The second stiffness may comprise a variable stiffness range between the first and third stiffnesses. The medial portion may extend from between 7-13 cm from a distal tip of the elongated catheter body and 25-35 cm from the distal tip of the elongated catheter body. The second stiffness variable range of the medial portion may increase in a distal-to-proximal direction at a substantially linear rate. An entirety of the second stiffness variable range of the medial portion may increase at a rate that does not exceed 60 gf/mm per cm of catheter body length in a distal-to-proximal direction (e.g., at a rate between 10-60 gf/mm per cm).

As an example, a catheter may comprise an elongated catheter body including a proximal portion, a medial portion, and a distal portion. The medial portion may be between the proximal portion and the distal portion. The medial portion may increase between a first stiffness and a second stiffness. The first stiffness may be located at a distal end of the medial portion and the second stiffness may be at a proximal end of the medial portion.

The location of the proximal and distal ends of the medial portion may vary in different examples. The distal end of the medial portion may be located between approximately 0.01 cm from a distal tip of the distal portion and 15 cm from a distal tip of the distal portion. The distal end of the medial portion may be located between approximately 5 cm from the distal tip of the distal portion and 10 cm from the distal tip of the distal portion. The proximal end of the medial portion may be located between approximately 20 cm from the distal tip of the distal portion and 40 cm from the distal tip of the distal portion. The proximal end of the medial portion may be located between approximately 25 cm from the distal tip of the distal portion and 35 cm from the distal tip of the distal portion. The distal end of the medial portion may be located approximately 10 cm from the distal tip of the distal portion. The proximal end of the medial portion may be located approximately 30 cm from the distal tip of the distal portion.

The respective first and second stiffnesses may vary in value in different examples. The first stiffness may be less than 50 gf/mm. The second stiffness may be greater than 400 gf/mm. The first stiffness may be between approximately 1 gf/mm and 100 gf/mm. The second stiffness may be between approximately 300 gf/mm and 600 gf/mm. Thus, the second stiffness may be at least five times greater than the first stiffness or, in other examples, at least two times greater than the first stiffness. The increase between the first and second stiffnesses may be substantially linear, or may be non-linear. The first stiffness may be between approximately 2 gf/mm and 10 gf/mm. The second stiffness may be approximately 500 gf/mm.

FIGS. 8 and 10 illustrate an example stiffness profile for a length of the catheter. As shown, the distal portion including the distal tip may have a first stiffness and the penultimate distal portion may have a second stiffness that is less than the first stiffness of the distal portion. Although FIGS. 8 and 10 illustrate specific stiffness values in gf/mm for illustrative purposes, it should be appreciated that the listed values are not meant to be limiting in scope. Additionally, the respective lengths of the distal and penultimate distal portions may vary in different examples.

Continuing to reference FIGS. 8 and 10, it can be seen that a penultimate proximal portion may be positioned adjacent to the penultimate distal portion in the proximal direction. The penultimate proximal portion may comprise a gradually increasing stiffness that ramps up along a length of the penultimate proximal portion. The increase in stiffness may be linear or non-linear, and segments of the length of the penultimate proximal portion may comprise substantially uniform stiffnesses. The penultimate proximal portion may transition into the proximal portion along which the stiffness may be substantially uniform up to and including the proximal end of the catheter.

With reference to FIGS. 8 and 10, it can be seen that an approximate length of the distal portion may be between about 0.5 cm and 5.0 cm, though greater or lesser lengths may be utilized. For example, the approximate length of the distal portion may be between 0.01 cm and 5 cm or, in other examples, between 0.01 cm and 0.2 cm.

The penultimate distal portion may have an approximate length between about 5 cm and 10 cm, though greater or lesser lengths may be utilized. The penultimate proximal portion may have an approximate length between about 15 cm and 40 cm, though greater or lesser lengths may be utilized. The proximal portion may have an approximate length between about 70 cm and 130 cm, though greater or lesser lengths may be utilized. The overall length of the catheter, including the distal, penultimate distal, penultimate proximal, and proximal portions may have a length of about 100 cm to 200 cm, though greater or lesser lengths may be utilized.

FIGS. 11-13 illustrate that the stiffnesses of each section of the catheter should not be construed as limited to the specific, exemplary values shown in FIGS. 8-10 by illustrating example ranges of values for such stiffnesses. However, it should also be appreciated that, in some examples, the stiffnesses of one or more portions may be outside of the exemplary ranges shown in FIGS. 11-13.

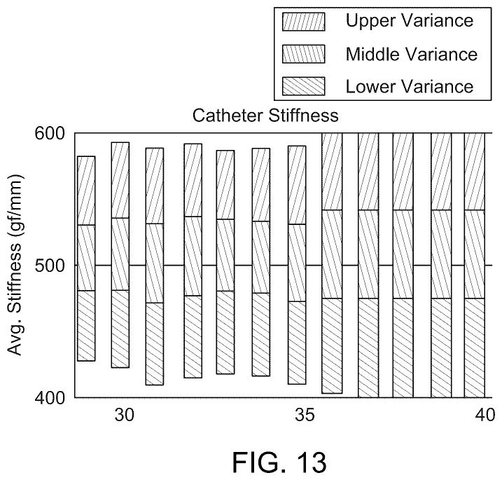

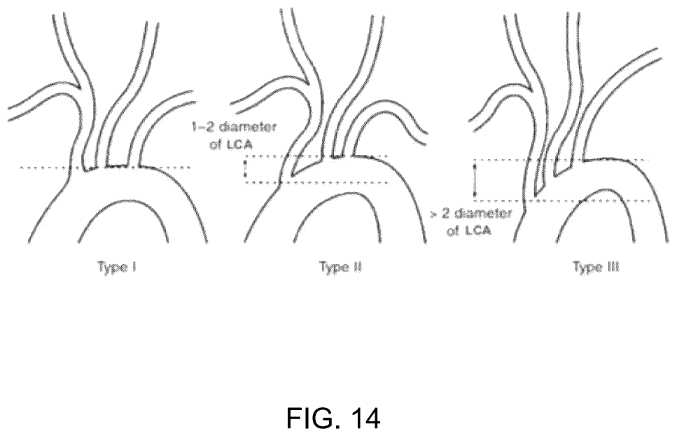

FIG. 11 illustrates ranges of stiffnesses along the penultimate proximal portion of the catheter along which the stiffness may ramp up. FIG. 12 illustrates ranges of stiffnesses along the penultimate distal portion, showing that at least a segment of the penultimate distal portion may comprise a substantially uniform stiffness. FIG. 13 illustrates ranges of stiffnesses along a proximal segment of the penultimate proximal portion and a distal segment of the proximal portion, illustrating how the ramping up stiffness segment transitions into a uniform stiffness segment.

FIGS. 11-13 illustrate that the ranges of stiffnesses may be considered as separated into an “upper variance”, a “middle variance”, and a “lower variance”. These delineations are merely for illustrative purposes and should not be construed as limiting the scope in any manner. It should not be construed that the use of an “upper variance” in one portion (e.g., the penultimate distal portion) requires the use of the same “upper variance” in another portion (e.g., the penultimate proximal portion). Thus, as an example, the penultimate distal portion may comprise stiffnesses in the upper variance range while the penultimate proximal portion may comprise stiffnesses in the middle variance ranges. Any of the variance ranges may be mixed-and-matched in certain examples.

The manner by which the stiffnesses along the length of the catheter may vary in different examples. As previously discussed, example methods or devices for varying the stiffnesses may include, but are not limited to, use of an outer jacket, coiled wire, braided mesh tubular layer, inner liner, and/or radiopaque marker. In an example, all or a portion of the length of the catheter, including the penultimate distal and distal portions, may be integrally formed from a unitary body and thus be continuous.

Claim Bank

Clause 1. A catheter comprising: an elongated catheter body having an outer jacket; a first outer jacket segment located within a distal portion of the elongated catheter body and having a first stiffness; and, a second outer jacket segment located proximally adjacent to the first outer jacket segment and having a second stiffness lower than the first outer jacket segment.

Clause 2. The catheter of clause 1, further comprising a third outer jacket segment located proximally adjacent to the second outer jacket segment and having a third stiffness lower than the second outer jacket segment.

Clause 3. The catheter of clause 2, further comprising a fourth outer jacket segment located proximally adjacent to the third outer jacket segment and having a fourth stiffness lower than the third outer jacket segment.

Clause 4. The catheter of clause 3, wherein the first outer jacket segment comprises a durometer within an inclusive range of 20 to 40 Shore A.

Clause 5. The catheter of clause 4, wherein the second outer jacket segment comprises a durometer within an inclusive range of 7 to 18 Shore A.

Clause 6. The catheter of clause 5, wherein the third outer jacket segment comprises a durometer within an inclusive range of 14 to 25 Shore A.

Clause 7. The catheter of clause 6, wherein the fourth outer jacket segment comprises a durometer within an inclusive range of 20 to 40 Shore A.

Clause 8. The catheter of clause 1, wherein the distal portion of the elongated catheter body further comprises a coil having a larger pitch near its distal end and a smaller pitch near its proximal end.

Clause 9. The catheter of clause 8, wherein the larger pitch of the coil is within an inclusive range of 0.006 inch to 0.01 inch.

Clause 10. The catheter of clause 9, wherein the smaller pitch of the coil is within an inclusive range of 0.003 inch to 0.0055 inch.

Clause 11. The catheter of clause 8, further comprising a braided nitinol tube disposed over the coil and having a smaller PPI near its distal end and a larger PPI near its proximal end.

Clause 12. The catheter of clause 11, wherein the smaller PPI is within an inclusive range of 55 PPI to 100 PPI.

Clause 13. A catheter comprising: an elongated catheter body; and, an outer jacket located along the elongated catheter body; wherein a distal region of the outer jacket comprises a plurality of jacket segments that decrease in hardness and then increase in hardness toward a distal end of the elongated catheter body.

Clause 14. The catheter of clause 13, wherein a distal-most jacket segment of the plurality of jacket segments has a higher hardness than a proximally-adjacent jacket segment of the plurality of jacket segments.

Clause 15. The catheter of clause 13, wherein the distal region of the elongated catheter body further comprises a coil having a larger pitch near its distal end and a smaller pitch near its proximal end.

Clause 16. The catheter of clause 15, further comprising a braided nitinol tube disposed over the coil and having a smaller PPI near its distal end and a larger PPI near its proximal end.

Clause 17. The catheter of clause 16, wherein the plurality of outer jacket segments is composed of a polyurethane elastomer.

Clause 18. The catheter of clause 17, further comprising an inner liner forming an inner passage through the catheter; the inner liner comprising a proximal PTFE tube and a distal polyolefin elastomer tube that are connected via a tube bonded over a distal end of the proximal PTFE tube and a proximal end of the distal polyolefin elastomer tube.

Clause 19. The catheter of clause 18, wherein the distal polyolefin elastomer tube has a length within an inclusive range of 11 cm to 15 cm, and wherein the proximal PTFE tube has a length within an inclusive range of 90 cm to 120 cm.

Clause 20. A catheter comprising: a catheter body means; and, an outer jacket means for providing a decrease in hardness and then an increase in hardness toward a distal end of the catheter body.

Clause 21. A catheter comprising: an elongated catheter body having an outer jacket; a first outer jacket segment located within a distal portion of the elongated catheter body and having a first hardness; and, a second outer jacket segment located proximally adjacent to the first outer jacket segment and having a second hardness lower than the first outer jacket segment.

Clause 22. The catheter of clause 21, wherein the first outer jacket segment comprises a durometer within an inclusive range of 20 to 40 Shore A.

Clause 23. The catheter of clause 22, wherein the first outer jacket segment comprises a durometer of 20, 21, 22, 23, 24, 25, 26, 27, 28, 29, 30, 31, 32, 33, 34, 35, 36, 37, 38, 39, or 40 Shore A.

Clause 24. The catheter of any of the clauses 21-23, wherein the second outer jacket segment comprises a durometer of 7, 8, 9, 10, 11, 12, 13, 14, 15, 16, 17, or 18 Shore A.

Clause 25. The catheter of any of the clauses 21-24, further comprising a third outer jacket segment located proximally adjacent to the second outer jacket segment and having a third hardness higher than the second outer jacket segment.

Clause 26. The catheter of any of the clauses 21-25, wherein the third outer jacket segment comprises a durometer of 14, 15, 16, 17, 18, 19, 20, 21, 22, 23, 24, or 25.

Clause 27. The catheter of any of the clauses 21-26, further comprising a fourth outer jacket segment located proximally adjacent to the third outer jacket segment and having a fourth stiffness higher than the third outer jacket segment.

Clause 28. The catheter of any of the clauses 21-27, wherein the fourth outer jacket segment comprises a durometer of 20, 21, 22, 23, 24, 25, 26, 27, 28, 29, 30, 31, 32, 33, 34, 35, 36, 37, 38, 39, or 40.

Clause 29. The catheter of any of the clauses 21-28, further comprising a fifth outer jacket segment located proximally adjacent to the fourth outer jacket segment and having a fifth hardness higher than the fourth outer jacket segment.

Clause 30. The catheter of any of the clauses 21-29, wherein the fifth outer jacket segment comprises a durometer of 30, 31, 32, 33, 34, 35, 36, 37, 38, 39, 40, 41, 42, 43, 44, 45, 46, 47, 48, 49, 50, 51, 52, 53, 54, or 55.

Clause 31. The catheter of any of the clauses 21-30, further comprising a sixth outer jacket segment located proximally adjacent to the fifth outer jacket segment and having a sixth hardness higher than the fifth outer jacket segment.

Clause 32. The catheter of any of the clauses 21-31, wherein the sixth outer jacket segment comprises a durometer of 45, 46, 47, 48, 49, 50, 51, 52, 53, 54, 55, 56, 57, 58, 59, 60, 61, 62, 63, 64, or 65.

Clause 33. The catheter of any of the clauses 21-32, further comprising a seventh outer jacket segment located proximally adjacent to the sixth outer jacket segment and having a seventh hardness higher than the sixth outer jacket segment.

Clause 34. The catheter of any of the clauses 21-33, wherein the seventh outer jacket segment comprises a durometer of 55, 56, 57, 58, 59, 60, 61, 62, 63, 64, 65, 66, 67, 68, 69, 70, 71, 72, 73, 74, 75, 76, 77, 78, 79, or 80.

Clause 35. The catheter of any of the clauses 21-34, wherein the elongated catheter body further comprises a coil having a larger pitch near its distal end and a smaller pitch near its proximal end.

Clause 36. The catheter of any of the clauses 21-35, wherein the larger pitch of the coil is within an inclusive range of 0.006 inch to 0.01 inch.

Clause 37. The catheter of any of the clauses 21-36, wherein the larger pitch of the coil is 0.006, 0.007, 0.008, 0.009, 0.01 inch.

Clause 38. The catheter of any of the clauses 21-37, wherein the smaller pitch of the coil is within an inclusive range of 0.003 inch to 0.0055 inch.

Clause 39. The catheter of any of the clauses 21-38, wherein the coil further has an intermediate pitch between the smaller pitch and the larger pitch, wherein the intermediate pitch is within an inclusive range of 0.003 to 0.008 inch.

Clause 40. The catheter of any of the clauses 21-39, wherein the intermediate pitch is 0.003, 0.004, 0.005, 0.006, 0.007, or 0.008.

Clause 41. The catheter of any of the clauses 21-40, further comprising a braided nitinol tube disposed over the coil and having a smaller PPI near its distal end and a larger PPI near its proximal end.

Clause 42. The catheter of any of the clauses 21-41, wherein the smaller PPI is within an inclusive range of 55 PPI to 100 PPI and wherein the larger PPI is within an inclusive range of 120 to 145.

Clause 43. The catheter of any of the clauses 21-42, wherein the smaller PPI is 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 PPI.

Clause 44. The catheter of any of the clauses 21-43, wherein the larger PPI is 120, 121, 122, 123, 124, 125, 126, 127, 128, 128, 130, 131, 132, 133, 134, 135, 136, 137, 138, 139, 140, 141, 142, 143, 144, or 145 PPI.

Clause 45. The catheter of any of the clauses 21-44, wherein any of the outer jacket segments are composed of a polyurethane elastomer.

Clause 46. The catheter of any of the clauses 21-45, further comprising an inner liner forming an inner passage through the catheter; the inner liner comprising a proximal PTFE tube and a distal polyolefin elastomer tube that are connected via a tube bonded over a distal end of the proximal PTFE tube and a proximal end of the distal polyolefin elastomer tube.

Clause 47. The catheter of any of the clauses 21-46, wherein the distal polyolefin elastomer tube has a length within an inclusive range of 11 cm to 15 cm, and wherein the proximal PTFE tube has a length within an inclusive range of 90 cm to 120 cm.

Clause 48. A catheter comprising: an elongated catheter body having an inner liner forming a passage through the catheter; the inner liner having a proximal portion formed of a first material and a distal portion formed of a second material; a coil disposed over a distal portion of the inner liner, wherein the coil has a larger pitch near its distal end and a smaller pitch near its proximal end.

Clause 49. The catheter of clause 48, wherein the transitions from the larger pitch to the smaller pitch across an interface between the first material and the second material of the inner liner.

Clause 50. The catheter of any of the clauses 48-49, wherein the larger pitch of the coil is within an inclusive range of 0.006 inch to 0.01 inch.

Clause 51. The catheter of any of the clauses 48-50, wherein the larger pitch of the coil is 0.006, 0.007, 0.008, 0.009, 0.01 inch.

Clause 52. The catheter of any of the clauses 48-51, wherein the smaller pitch of the coil is within an inclusive range of 0.003 inch to 0.0055 inch.

Clause 53. The catheter of any of the clauses 48-52, wherein the coil further has an intermediate pitch between the smaller pitch and the larger pitch, wherein the intermediate pitch is within an inclusive range of 0.003 to 0.008 inch.

Clause 54. The catheter of any of the clauses 48-53, wherein the intermediate pitch is 0.003, 0.004, 0.005, 0.006, 0.007, or 0.008.

Clause 55. The catheter of any of the clauses 48-54, further comprising a braided nitinol tube disposed over the coil and having a smaller PPI near its distal end and a larger PPI near its proximal end.

Clause 56. The catheter of any of the clauses 48-55, wherein the smaller PPI is within an inclusive range of 55 PPI to 100 PPI and wherein the larger PPI is within an inclusive range of 120 to 145.

Clause 57. The catheter of any of the clauses 48-56, wherein the smaller PPI is 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 PPI.

Clause 58. The catheter of any of the clauses 48-57, wherein the larger PPI is 120, 121, 122, 123, 124, 125, 126, 127, 128, 128, 130, 131, 132, 133, 134, 135, 136, 137, 138, 139, 140, 141, 142, 143, 144, or 145 PPI.

Clause 59. A catheter comprising: an elongated catheter body having an outer jacket; a first outer jacket segment located within a distal portion of the elongated catheter body and having a first durometer; and, a second outer jacket segment located proximally adjacent to the first outer jacket segment and having a second durometer lower than the first outer jacket segment; wherein the durometer of the first outer jacket segment is 100, 110, 120, 130, 140, 150, 160, 170, 180, 190, 200, 210, 220, 230, 240, 250, 260, 270, 280, 290, 300, 310, 320, 330, 340, or 350 percent higher than the second outer jacket segment.

Clause 60. A catheter comprising: an elongated catheter body having a plurality of layers forming a catheter wall; wherein the catheter wall forms a lumen between a proximal and a distal end of the catheter; and, wherein a distal portion of the catheter increases in flexibility toward the distal end of the catheter and then decreases in flexibility until the distal end of the catheter.

Clause 61. A catheter comprising: an elongated catheter body; and, an outer jacket located along the elongated catheter body having a distal region with; wherein the distal region decreases in hardness toward a distal end of the catheter body and then increases in hardness until the distal end of the catheter body.

Clause 62. The catheter of clause 61, wherein the distal region increases in hardness within a length extending between about 0 to 0.5 cm from the distal end of the catheter body and to a hardness within an inclusive range of 20 A to 40 A Shore.

Clause 62. The catheter of clause 61, wherein the distal region increases in hardness within a length extending between about 0 to 3 cm from the distal end of the catheter body and increases to a hardness within an inclusive range of 20 A to 40 A Shore.

Clause 63. The catheter of clause 62, wherein the distal region decreases in hardness within a length extending between at least about 0.5 to 15 cm from the distal end of the catheter body and decreases to a hardness within an inclusive range of 12-18 Shore A.

Clause 64. A catheter comprising: an elongated catheter body having a distal region with a distal section and a proximal section; wherein the distal section has a stiffness that is 100 (i.e., double), 110, 120, 130, 140, 150, 160, 170, 180, 190, 200, 210, 220, 230, 240, 250, 260, 270, 280, 290, 300 (i.e., triple), 310, 320, 330, 340, 350 percent higher relative to the proximal section.

Clause 65. A catheter comprising: an elongated catheter body having a distal region and a proximal region; wherein the proximal region has a stiffness that is 7,000, 7,500, 8,000, 8,500, 9,000, 9,500, 10,000, 10,500, 11,000 percent, or percent values within an inclusive range of those percentages higher than the distal region.

Clause 66. The catheter of clause 65, wherein the distal region is between about 0 to 7-12 cm proximal of the distal tip; and wherein the proximal region is between 7-12 cm proximal of the distal tip and 40-100 cm from the distal tip.

Clause 67. A catheter comprising: an elongated catheter body having a distal end; wherein between a distal end of the catheter and about 10 cm proximal of the distal end, an average stiffness is between about 2-10 gf/mm, between about 10 cm and 13 cm from the distal tip, the average stiffness proximally increases between about 9 gf/mm to 39 gf/mm, between about 13 cm and 30 cm from the distal tip, the average stiffness proximally increases between about 29 gf/mm to 480, and between about 30 cm and 40 cm from the distal tip, the average stiffness proximally increases between about 480 gf/mm to 495 gf/mm.

Clause 68. A catheter comprising: an elongated catheter body having an outer jacket; a first outer jacket segment located within a distal portion of the elongated catheter body and having a first stiffness; and, a second outer jacket segment proximally to the first outer jacket segment and having a second stiffness lower than the first outer jacket segment.

Clause 69. The catheter of clause 68, further comprising a third outer jacket segment proximally abutting the second outer jacket segment and having a third stiffness higher than the second outer jacket segment.

Clause 70. The catheter of the clauses 68-69, further comprising a fourth outer jacket segment located proximally adjacent to the third outer jacket segment and having a fourth stiffness higher than the third outer jacket segment.

Clause 71. The catheter of any of the clauses 68-70, wherein the first outer jacket segment comprises a durometer within an inclusive range of 20 to 40 Shore A.

Clause 72. The catheter of any of the clauses 68-71, wherein the second outer jacket segment comprises a durometer within an inclusive range of 7 to 18 Shore A.

Clause 73. The catheter of any of the clauses 68-72, wherein a third outer jacket segment comprises a durometer within an inclusive range of 14 to 25 Shore A.

Clause 74. The catheter of any of the clauses 68-73, wherein a fourth outer jacket segment comprises a durometer within an inclusive range of 20 to 40 Shore A.

Clause 75. The catheter of any of the clauses 68-74, wherein the elongated catheter body further comprises a coil having a larger pitch near its distal end and a smaller pitch near its proximal end.

Clause 76. The catheter of any of the clauses 68-75, wherein the larger pitch of the coil is within an inclusive range of 0.006 inch to 0.01 inch.

Clause 77. The catheter of any of the clauses 68-76, wherein the smaller pitch of the coil is within an inclusive range of 0.003 inch to 0.0055 inch.

Clause 78. The catheter of any of the clauses 68-77, further comprising a braided nitinol tube disposed over the coil and having a smaller PPI near its distal end and a larger PPI near its proximal end.

Clause 79. The catheter of any of the clauses 68-78, wherein the smaller PPI is within an inclusive range of 55 PPI to 100 PPI and wherein the larger PPI is within an inclusive range of 120 to 145.

Clause 80. A catheter comprising: an elongated catheter body; and, an outer jacket located along the elongated catheter body having a distal region with; wherein the distal region decreases in hardness toward a distal end of the catheter body and then increases in hardness until the distal end of the catheter body.

Clause 81. The catheter of clause 80, wherein the distal region increases in hardness within a length extending between about 0 to 0.5 cm from the distal end of the catheter body and to a hardness within an inclusive range of 20 A to 40 A Shore.

Clause 82. The catheter of the clauses 80-81, wherein the distal region increases in hardness within a length extending between about 0 to 3 cm from the distal end of the catheter body and increases to a hardness within an inclusive range of 20 A to 40 A Shore.

Clause 83. The catheter of any of the clauses 80-82, wherein the distal region decreases in hardness within a length extending between at least about 0.5 to 15 cm from the distal end of the catheter body and decreases to a hardness within an inclusive range of 12-18 Shore A.

Clause 84. The catheter of any of the clauses 80-83, wherein the distal region increases in hardness within a length extending between about 0 to 3 cm from the distal end and doubles or triples a Shore A hardness value proximally within the distal region.

Clause 85. The catheter of any of the clauses 80-84, further comprising an inner liner forming an inner passage through the catheter; the inner liner comprising a proximal PTFE tube and a distal polyolefin elastomer tube that are connected via a tube bonded over a distal end of the proximal PTFE tube and a proximal end of the distal polyolefin elastomer tube.

Clause 86. The catheter of any of the clauses 80-85, wherein the distal polyolefin elastomer tube has a length within an inclusive range of 11 cm to 15 cm, and wherein the proximal PTFE tube has a length within an inclusive range of 90 cm to 120 cm.

Clause 87. A catheter comprising: a catheter body means; and, an outer jacket means for providing a decrease in hardness and then an increase in hardness toward a distal end of the catheter body.

Clause 88. A catheter comprising: an elongated catheter body; a penultimate distal portion having a first stiffness; and a distal portion having a second stiffness, wherein the second stiffness is greater than the first stiffness.

Clause 89. The catheter of clause 88, wherein the catheter stiffness increases from a first stiffness at about 5 cm from a distal tip of the distal portion to a second stiffness at about 30 cm from a distal tip of the distal portion.

Clause 90. The catheter of the clauses 88-89, wherein the first stiffness is less than 50 gf/mm.

Clause 91. The catheter of any of the clauses 88-90, wherein the second stiffness is greater than 400 gf/mm.

Clause 92. The catheter of any of the clauses 88-91, wherein the stiffness increase is substantially linear.

Clause 93. The catheter of any of the clauses 88-92, wherein the stiffness at 30 cm from the distal tip is between 100 gf/mm and 400 gf/mm.

Clause 94. The catheter of any of the clauses 88-93, wherein the stiffness at 40 cm from the distal tip is between 100 gf/mm and 400 gf/mm.

Clause 95. The catheter of any of the clauses 88-94, wherein the stiffness at 30 cm from the distal tip is between 150 gf/mm and 350 gf/mm.

Clause 96. The catheter of any of the clauses 88-95, wherein the stiffness at 40 cm from the distal tip is between 150 gf/mm and 350 gf/mm.

Clause 97. The catheter of any of the clauses 88-96, wherein the stiffness from 40 cm to 90 cm from the distal tip is between 400 gf/mm and 600 gf/mm.

Clause 98. The catheter of any of the clauses 88-97, wherein the stiffness at 40 cm to 120 cm from the distal tip is between 400 gf/mm and 600 gf/mm.

Clause 99. The catheter of any of the clauses 88-98, further comprising a braided mesh tubular layer extending through at least a portion of the elongated catheter body, the braided mesh tubular layer having the first stiffness along the penultimate distal portion and the second stiffness along the distal portion.

Clause 100. The catheter of any of the clauses 88-99, wherein the braided mesh tubular layer is located within walls of the catheter.

Clause 101. The catheter of any of the clauses 88-100, wherein the braided mesh tubular layer is located between an outer jacket and an inner coil.

Clause 102. The catheter of any of the clauses 88-101, wherein the braided mesh tubular layer is located underneath an inner coil.

Clause 103. The catheter of any of the clauses 88-102, wherein a picks-per-inch of the braided mesh tubular layer along a first portion having the first stiffness is greater than a picks-per-inch of the braided mesh tubular layer along a second portion having the second stiffness.

Clause 104. The catheter of any of the clauses 88-103, wherein a diameter of the wire(s) forming the braided mesh tubular layer is greater along the second portion having the second stiffness than the first portion having the first stiffness.

Clause 105. The catheter of any of the clauses 88-98, further comprising an inner liner extending through at least a portion of the elongated catheter body, the inner liner having the first stiffness along the penultimate distal portion and the second stiffness along the distal portion.

Clause 106. The catheter of clause 105, wherein the inner liner comprises a first segment along the penultimate distal portion and a second segment along the distal portion, wherein the first segment is composed of a first material having a first hardness, and wherein the second segment is composed of a second material having a second hardness.

Clause 107. The catheter of any of the clauses 105-106, wherein the first hardness is less than the second hardness.

Clause 108. The catheter of any of the clauses 105-107, wherein the first segment is composed of a first material having a first flexibility, and wherein the second segment is composed of a second material having a second flexibility.

Clause 109. The catheter of any of the clauses 105-108, wherein the first flexibility is greater than the second flexibility.

Clause 110. The catheter of any of the clauses 105-109, wherein the first material is comprised of PTFE and wherein the second material is comprised of polyolefin elastomer.

Clause 111. The catheter of any of the clauses 105-110, wherein the first segment and the second segment are integrally formed from a unitary body.

Clause 112. A catheter comprising: an elongated catheter body; and, an outer jacket located along the elongated catheter body having a distal region; wherein the distal region has a variable hardness that, in a distal direction along a longitudinal axis, decreases in hardness toward a distal end of the catheter body and then increases in hardness until the distal-most end of the catheter body.

Clause 113. The catheter of the clause 112, wherein the hardness of the outer jacket at the distal-most end of the catheter body is between 20 A and 50 A Shore.

Clause 114. The catheter of any of the clauses 112-113, wherein the hardness of the outer jacket at a portion of the outer jacket proximal to the distal-most end of the catheter body is between 10 A and 20 A Shore.

Clause 115. The catheter of any of the clauses 112-114, wherein the distal region having the variable hardness decreasing in hardness toward a distal end of the catheter body comprises a first outer jacket segment having a first hardness bonded at an interface region to a second outer jacket segment having a second hardness.

Clause 116. The catheter of any of the clauses 112-115, wherein the interface region has a smooth transition from the first hardness to the second hardness.

Clause 117. The catheter of any of the clauses 112-116, wherein the interface region is at least 0.5 cm in length.

Clause 118. A catheter comprising: an elongated catheter body; a proximal portion; a medial portion; and a distal portion; wherein the medial portion is between the proximal portion and the distal portion; and wherein the medial portion increases between a first stiffness and a second stiffness.

Clause 119. The catheter of the clause 118, wherein the first stiffness is at a distal end of the medial portion and wherein the second stiffness is at a proximal end of the medial portion.

Clause 120. The catheter of any of the clauses 118-119, wherein the distal end of the medial portion is located between approximately 0.01 cm from a distal tip of the distal portion and 15 cm from a distal tip of the distal portion.

Clause 121. The catheter of any of the clauses 118-120, wherein the distal end of the medial portion is located between approximately 5 cm from the distal tip of the distal portion and 10 cm from the distal tip of the distal portion.

Clause 122. The catheter of any of the clauses 118-121, wherein the proximal end of the medial portion is located between approximately 20 cm from the distal tip of the distal portion and 40 cm from the distal tip of the distal portion.

Clause 123. The catheter of any of the clauses 118-122, wherein the proximal end of the medial portion is located between approximately 25 cm from the distal tip of the distal portion and 35 cm from the distal tip of the distal portion.

Clause 124. The catheter of any of the clauses 118-123, wherein the distal end of the medial portion is located approximately 10 cm from the distal tip of the distal portion.

Clause 125. The catheter of any of the clauses 118-124, wherein the proximal end of the medial portion is located approximately 30 cm from the distal tip of the distal portion.

Clause 126. The catheter of any of the clauses 118-125, wherein the first stiffness is less than 50 gf/mm.

Clause 127. The catheter of any of the clauses 118-126, wherein the second stiffness is greater than 400 gf/mm.

Clause 128. The catheter of any of the clauses 118-127, wherein the first stiffness is between approximately 1 gf/mm and 100 gf/mm.

Clause 129. The catheter of any of the clauses 118-128, wherein the second stiffness is between approximately 300 gf/mm and 600 gf/mm.

Clause 130. The catheter of any of the clauses 118-129, wherein the second stiffness is at least five times greater than the first stiffness.

Clause 131. The catheter of any of the clauses 118-130, wherein the second stiffness is at least two times greater than the first stiffness.

Clause 132. The catheter of any of the clauses 118-131, wherein an increase between the first stiffness and the second stiffness is substantially linear.

Clause 133. The catheter of any of the clauses 118-132, wherein the increase between the first stiffness and the second stiffness is non-linear.

Clause 134. The catheter of any of the clauses 118-133, wherein the second stiffness is approximately 500 gf/mm.

Clause 135. The catheter of any of the clauses 118-134, wherein the first stiffness is between approximately 2 gf/mm and 10 gf/mm.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A catheter comprising:
an elongated catheter body;
a penultimate distal portion having a first stiffness; and
a distal portion having a second stiffness, wherein the second stiffness is greater than the first stiffness.

2. The catheter of claim 1, wherein the distal portion comprises a length of between 0.01 cm and 5 cm.

3. The catheter of claim 2, wherein the distal portion comprises a length between 0.01 cm and 0.2 cm.

4. The catheter of claim 1, wherein a stiffness of the elongated catheter body increases from a lower stiffness at a first point about 10 cm from a distal tip of the distal portion to a greater stiffness at a second point about 30 cm from a distal tip of the distal portion.

5. The catheter of claim 4, wherein the lower stiffness is less than 50 gf/mm.

6. The catheter of claim 5, wherein the greater stiffness is greater than 400 gf/mm.

7. The catheter of claim 4, wherein the greater stiffness is at least five times greater than the lower stiffness.

8. The catheter of claim 4, wherein an increase between the lower stiffness and the greater stiffness is substantially linear.

9. The catheter of claim 1, wherein a stiffness at a point 20 cm proximally from a distal tip of the distal portion is between 100 gf/mm and 400 gf/mm.

10. The catheter of claim 9, wherein a stiffness at a point 20 cm proximally from the distal tip of the distal portion is between 150 gf/mm and 350 gf/mm.

11. The catheter of claim 1, wherein a stiffness along a segment between 30 cm and 90 cm proximally from a distal tip of the distal portion is between 400 gf/mm and 600 gf/mm.

12. The catheter of claim 1, wherein a stiffness along a segment between 30 cm and 120 cm proximally from a distal tip of the distal portion is between 400 gf/mm and 600 gf/mm.

13. The catheter of claim 1, wherein the penultimate distal portion and the distal portion are integrally formed from a unitary body.

14. The catheter of claim 1, wherein the penultimate distal portion comprises a first outer jacket segment having the first stiffness and wherein the distal portion comprises a second outer jacket segment having the second stiffness.

15. The catheter of claim 1, further comprising a coiled wire extending through at least a portion of the elongated catheter body, the coiled wire having the first stiffness along the penultimate distal portion and the second stiffness along the distal portion.

16. The catheter of claim 15, wherein the coiled wire comprises a first spacing between coils along the penultimate distal portion and a second spacing between coils along the distal portion, the first spacing being less than the second spacing.

17. The catheter of claim 15, wherein the coiled wire comprises a first wire diameter along the penultimate distal portion and a second wire diameter along the distal portion, the first wire diameter being greater than the second wire diameter.

18. The catheter of claim 1, further comprising a braided mesh tubular layer extending through at least a portion of the elongated catheter body, the braided mesh tubular layer having the first stiffness along the penultimate distal portion and the second stiffness along the distal portion.

19. The catheter of claim 1, further comprising an inner liner extending through at least a portion of the elongated catheter body, the inner liner having the first stiffness along the penultimate distal portion and the second stiffness along the distal portion.

20. A catheter comprising:
an elongated catheter body having an outer jacket;
a first outer jacket segment located at a distal-most portion of the elongated catheter body and having a first stiffness; and,
a second outer jacket segment located at a second-most distal portion of the first outer jacket segment and having a second stiffness lower than the first stiffness.

21. A catheter comprising:
an elongated catheter body; and,
an outer jacket located along the elongated catheter body having a distal region; wherein the distal region has a variable hardness that, in a distal direction along a longitudinal axis, decreases in hardness toward a distal end of the catheter body and then increases in hardness until a distal-most end of the catheter body.

22. The catheter of claim 21, wherein the hardness of the outer jacket at the distal-most end of the catheter body is between 20 A and 50 A Shore.

23. The catheter of claim 22, wherein the hardness of the outer jacket at a portion of the outer jacket proximal to the distal-most end of the catheter body is between 10 A and 20 A Shore.

24. The catheter of claim 21, wherein the distal region having the variable hardness decreasing in hardness toward a distal end of the catheter body comprises a first outer jacket segment having a first hardness bonded at an interface region to a second outer jacket segment having a second hardness.

25. The catheter of claim 24, wherein the interface region has a smooth transition from the first hardness to the second hardness.

26. The catheter of claim 25, wherein the interface region is at least 0.1 cm in length.

27. A catheter comprising:
an elongated catheter body having a distal portion, a proximal portion, and a medial portion therebetween;
wherein the distal portion has a first stiffness that is less than 50 gf/mm, wherein the medial portion has a second stiffness, and the proximal portion has a third stiffness that is greater than 400 gf/mm;
wherein the second stiffness is a variable stiffness range between the first stiffness and the third stiffness;
wherein the medial portion extends from between 7-13 cm from a distal tip of the elongated catheter body and 25-35 cm from the distal tip of the elongated catheter body.

28. The catheter of claim 27, wherein the second stiffness variable range of the medial portion increases in a distal-to-proximal direction at a substantially linear rate.

29. The catheter of claim 27, wherein an entirety of the second stiffness variable range of the medial portion increases at a rate that does not exceed 60 gf/mm per cm of catheter body length in a distal-to-proximal direction.

30. The catheter of claim 27, wherein an entirety of the second stiffness variable range of the medial portion increases at a rate between 10-60 gf/mm per cm of catheter body length in a distal-to-proximal direction.
